SEQUENCE LISTING (23 sequences)

SEQ ID NO: 1 - protein, 21 aa, synthetic construct
  source 1..21: note = Ciltacabtagene autoleucel CAR CD8a signal peptide, CD8a SP amino acid sequence
MALPVTALLLPLALLLHAARP

SEQ ID NO: 2 - protein, 119 aa, synthetic construct
  source 1..119: note = Ciltacabtagene autoleucel CAR BCMA binding domain, VHH1 amino acid sequence
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSS

SEQ ID NO: 3 - protein, 5 aa, synthetic construct
  source 1..5: note = Ciltacabtagene autoleucel CAR BCMA binding domain, G4S linker amino acid sequence
GGGGS

SEQ ID NO: 4 - protein, 118 aa, synthetic construct
  source 1..118: note = Ciltacabtagene autoleucel CAR BCMA binding domain, VHH2 amino acid sequence
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 5 - protein, 45 aa, synthetic construct
  source 1..45: note = Ciltacabtagene autoleucel CAR CD8a hinge amino acid sequence
TTTPAPRPPTPAPTIASQPLSLRPEACRPAAGGAVHTRGLDFACD

SEQ ID NO: 6 - protein, 24 aa, synthetic construct
  source 1..24: note = Ciltacabtagene autoleucel CAR CD8a transmembrane amino acid sequence
IYIWAPLAGTCGVLLLSLVITLYC

SEQ ID NO: 7 - protein, 42 aa, synthetic construct
  source 1..42: note = Ciltacabtagene autoleucel CAR CD137 Cytoplasmic amino acid sequence
KRGRKKLLYIFKQPFMRPVQTTQEEDGCSCRFPEEEEGGCEL

SEQ ID NO: 8 - protein, 112 aa, synthetic construct
  source 1..112: note = Ciltacabtagene autoleucel CAR CD3z Cytoplasmic amino acid sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 63 bp, synthetic construct
  source 1..63: mol_type = other DNA; note = Ciltacabtagene autoleucel CAR CD8a signal peptide CD8a SP nucleic acid sequence
atggctctgcccgtcaccgctctgctgctgcctctggctctgctgctgcacgctgctcgccct

SEQ ID NO: 10 - DNA, 357 bp, synthetic construct
  source 1..357: mol_type = other DNA; note = Ciltacabtagene autoleucel CAR BCMA binding domain, VHH1 nucleic acid sequence
caggtcaaactggaagaatctggcggaggcctggtgcaggcaggacggagcctgcgcctgagctgcgcagcatccgagcacaccttcagctcccacgtgatgggctggtttcggcaggccccaggcaaggagagagagagcgtggccgtgatcggctggagggacatctccacatcttacgccgattccgtgaagggccggttcaccatcagccgggacaacgccaagaagacactgtatctgcagatgaacagcctgaagcccgaggacaccgccgtgtactattgcgcagcaaggagaatcgacgcagcagactttgattcctggggccagggcacccaggtgacagtgtctagc

SEQ ID NO: 11 - DNA, 15 bp, synthetic construct
  source 1..15: mol_type = other DNA; note = Ciltacabtagene autoleucel CAR BCMA binding domain, G4S linker nucleic acid sequence
ggaggaggaggatct

SEQ ID NO: 12 - DNA, 354 bp, synthetic construct
  source 1..354: mol_type = other DNA; note = Ciltacabtagene autoleucel CAR BCMA binding domain, VHH2 nucleic acid sequence
gaggtgcagctggtggagagcggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagagcgtgaagggcaggtttaccatctcccgcgataacgccaagaatacagtggtgctgcagatgaactccctgaaacctgaggacacagccctgtactattgtgccgccgatcggaagagcgtgatgagcattagaccagactattgggggcagggaacacaggtgaccgtgagcagc

SEQ ID NO: 13 - DNA, 135 bp, synthetic construct
  source 1..135: mol_type = other DNA; note = Ciltacabtagene autoleucel CAR CD8a hinge nucleic acid sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 72 bp, synthetic construct
  source 1..72: mol_type = other DNA; note = Ciltacabtagene autoleucel CAR CD8a transmembrane nucleic acid sequence
atctacatctgggcgcccttggccgggacttgtggggtccttctcctgtcactggttatcaccctttactgc

SEQ ID NO: 15 - DNA, 126 bp, synthetic construct
  source 1..126: mol_type = other DNA; note = Ciltacabtagene autoleucel CAR CD137 Cytoplasmic nucleic acid sequence
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 16 - DNA, 339 bp, synthetic construct
  source 1..339: mol_type = other DNA; note = Ciltacabtagene autoleucel CAR CD3z Cytoplasmic nucleic acid sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 17 - protein, 488 aa, synthetic construct
  source 1..488: note = Ciltacabtagene autoleucel CAR amino acid sequence
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 5 aa, synthetic construct
  source 1..5: note = Ciltacabtagene autoleucel CAR BCMA binding domain, VHH1 CDR1
SHVMG

SEQ ID NO: 19 - protein, 17 aa, synthetic construct
  source 1..17: note = Ciltacabtagene autoleucel CAR BCMA binding domain, VHH1 CDR2
VIGWRDISTSYADSVKG

SEQ ID NO: 20 - protein, 11 aa, synthetic construct
  source 1..11: note = Ciltacabtagene autoleucel CAR BCMA binding domain, VHH1 CDR3
ARRIDAADFDS

SEQ ID NO: 21 - protein, 5 aa, synthetic construct
  source 1..5: note = Ciltacabtagene autoleucel CAR BCMA binding domain, VHH2 CDR1
TFTMG

SEQ ID NO: 22 - protein, 17 aa, synthetic construct
  source 1..17: note = Ciltacabtagene autoleucel CAR BCMA binding domain, VHH2 CDR2
AISLSPTLAYYAESVKG

SEQ ID NO: 23 - protein, 13 aa, synthetic construct
  source 1..13: note = Ciltacabtagene autoleucel CAR BCMA binding domain, VHH2 CDR3
ADRKSVMSIRPDY

(19) United States
(12) Patent Application Publication
(10) Pub. No.: US 2024/0156962 A1
(43) Pub. Date: May 16, 2024

(54) BCMA-TARGETED CAR-T CELL THERAPY OF MULTIPLE MYELOMA

(71) Applicants: LEGEND BIOTECH USA INC., Somerset, NJ (US); JANSSEN BIOTECH, INC., Horsham, PA (US)

(72) Inventors: Lida PACAUD, Somerset, NJ (US); Muhammad AKRAM, Monroe, NJ (US); Dong GENG, Somerset, NJ (US); Jordan SCHECTER, Livingston, NJ (US); Carolyn Chang JACKSON, West Orange, NJ (US); Enrique ZUDAIRE UBANI, Ambler, PA (US); Deepu MADDURI, San Jose, CA (US)

(73) Assignees: LEGEND BIOTECH USA INC., Somerset, NJ (US); JANSSEN BIOTECH, INC., Horsham, PA (US)

(21) Appl. No.: 18/499,720

(22) Filed: Nov. 1, 2023

Related U.S. Application Data
(60) Provisional application No. 63/421,740, filed on Nov. 2, 2022.

(51) Int. Cl. A61K 39/00; A61P 35/00; C12Q 1/6886

(52) U.S. Cl. CPC A61K 39/464417; A61K 39/4611; A61K 39/4631; A61P 35/00; C12Q 1/6886; A61K 2239/38; A61K 2239/48; C12Q 2600/106; C12Q 2600/118

(57) ABSTRACT

A method for assessing responsiveness of a subject to a treatment comprising T cells expressing a bivalent BCMA-targeting chimeric antigen receptor (CAR), comprising administering to the subject the T cells, and assessing the responsiveness of the subject to the treatment based on time length the subject maintains minimal residual disease (MRD) negative status.

### CROSS-REFERENCE TO RELATED APPLICATIONS

This application claims benefit of priority of U.S. Provisional Patent Application No. 63/421,740 filed on Nov. 2, 2022, the disclosure of which is incorporated by reference herein in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14651-049-999_SEQ_LISTING.xml”, was created on Oct. 16, 2023, and is 28,402 bytes in size.

### 1. FIELD

Provided herein is a method for assessing responsiveness of a subject (e.g., a subject having multiple myeloma) to a treatment comprising T cells expressing a bivalent BCMA-targeting chimeric antigen receptor (CAR).

### 2. BACKGROUND

Multiple myeloma (MM) is an incurable aggressive plasma malignancy, which is categorized as a B-cell neoplasia and proliferates in uncontrollably method in the bone marrow, interfering with the normal metabolic production of blood cells and causing painful bone lesions (Garfall, A. L. et al., Discovery Med. 2014, 17, 37). Multiple myeloma can present clinically with hypercalcemia, renal insufficiency, anemia, bony lesions, bacterial infections, hyperviscosity, and amyloidosis (Robert Z. Orlowski, Cancer Cell. 2013, 24(3)). Antibody-based cell immunotherapies that target B-cell maturation antigen (BCMA) have recently demonstrated substantial clinical benefit for patients with multiple myeloma. However, there is a need in the art for meaningful clinical end point for effectively assessing patient response to such cell immunotherapies.

### 3. SUMMARY

In one aspect, provided here in is a method for assessing responsiveness of a subject to a treatment comprising T cells expressing a bivalent BCMA-targeting chimeric antigen receptor (CAR), comprising: (a) administering to the subject the T cells; (b) measuring time length the subject maintains minimal residual disease (MRD) negative status; and (c) assessing the responsiveness of the subject to the treatment based on that (i) the MRD negative status is maintained for shorter than 6 months; (ii) the MRD negative status is maintained for at least 6 months and shorter than 12 months; or (iii) the MRD negative status is maintained for at least 12 months.

In some embodiments, the bivalent BCMA-targeting CAR provided herein comprises an extracellular antigen binding domain comprising a first VHH domain and a second VHH domain, a transmembrane domain, and an intracellular signaling domain. In some embodiments, the first VHH domain comprising a CDR1, a CDR2, and a CDR3 as set forth in the VHH domain comprising the amino acid sequence of SEQ ID NO: 2, and the second VHH domain comprising a CDR1, a CDR2, and a CDR3 as set forth in the VHH domain comprising the amino acid sequence of SEQ ID NO: 4. In some embodiments, the first VHH domain comprises a CDR1 comprising the amino acid sequence of SEQ ID NO: 18, a CDR2 comprising the amino acid sequence of SEQ ID NO: 19, a CDR3 comprising the amino acid sequence of SEQ ID NO: 20; and the second VHH domain comprises a CDR1 comprising the amino acid sequence of SEQ ID NO: 21, a CDR2 comprising the amino acid sequence of SEQ ID NO: 22, and a CDR3 comprising the amino acid sequence of SEQ ID NO: 23. In some embodiments, the first VHH domain comprises the amino acid sequence of SEQ ID NO: 2 and the second VHH domain comprises the amino acid sequence of SEQ ID NO: 4.

In some embodiments, the first VHH domain is at the N-terminus of the second VHH domain, or the first VHH domain is at the C-terminus of the second VHH domain.

In some embodiments, the first VHH domain is linked to the second VHH domain via a linker comprising the amino acid sequence of SEQ ID NO: 3.

In some embodiments, the transmembrane domain is derived from a molecule selected from the group consisting of CD8a, CD4, CD28, CD137, CD80, CD86, CD152 and PD1.

In some embodiments, the transmembrane domain is derived from CD8a and comprises the amino acid sequence of SEQ ID NO: 6.

In some embodiments, the intracellular signaling domain comprises a primary intracellular signaling domain of an immune effector cell. In some embodiments, the primary intracellular signaling domain is derived from CD3ζ comprising the amino acid sequence of SEQ ID NO: 8.

In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and any combination thereof. In some embodiments, the co-stimulatory signaling domain comprises a cytoplasmic domain of CD137 comprising the amino acid sequence of SEQ ID NO: 7.

In some embodiments, the CAR provided herein further comprises a hinge domain located between the C-terminus of the extracellular antigen binding domain and the N-terminus of the transmembrane domain. In some embodiments, the hinge domain is derived from CD8a comprising the amino acid sequence of SEQ ID NO: 5.

In some embodiments, the CAR further comprises a signal peptide located at the N-terminus of the polypeptide. In some embodiments, the signal peptide is derived from CD8a comprising the amino acid sequence of SEQ ID NO: 1.

In some embodiments, the CAR provided herein comprises the amino acid sequence of SEQ ID NO: 17.

In some embodiments, the subject has a disease or disorder. In some embodiments, the disease or disorder is cancer. In some embodiments, the cancer is multiple myeloma. In some embodiments, the cancer is refractory or relapsed multiple myeloma.

In some embodiments, the method provided herein comprises obtaining bone marrow aspirate or biopsy from the subject for assessing MRD status. In some embodiments, MRD status is monitored using next generation sequencing (NGS) of bone marrow aspirate DNA. In some embodiments, the NGS is performed via clonoSEQ.

In some embodiments, baseline bone marrow aspirates are used to define the myeloma clones, and post-treatment samples are used to evaluate MRD negativity. In some embodiments, evaluable samples are those that passed one or more of, or all of, calibration, quality control, and sufficiency of cells evaluable at a particular sensitivity level. In some embodiments, the sensitivity level is about 10⁻⁶, 10⁻⁵, 10⁻⁴, or 10⁻³.

In some embodiments, the time length the subject maintains MRD negative status is measured from the time when MRD is first achieved in the subject.

In some embodiments, the method provided herein comprises assessing the likelihood of the subject to have complete response (CR), partial response (PR), stringent CR (sCR), or very good PR (VGPR).

In some embodiments, the method provided herein comprises determining that the subject is likely to have CR, PR, sCR, or VGPR if the MRD negative status is maintained for shorter than 6 months in the subject. In some embodiments, the method provided herein comprises determining that the subject is likely to have CR or sCR if the MRD negative status is maintained for longer than 6 months in the subject.

In some embodiments, the method provided herein comprises assessing the likelihood of the subject to have progression-free survival.

In some embodiments, the method provided herein comprises determining that the subject is likely to have progression-free survival for at least 12 months post the treatment if the MRD negative status is maintained for longer than 6 months in the subject.

In some embodiments, the method provided herein comprises determining that the subject is likely to have progression-free survival for at least 24 months post the treatment if the MRD negative status is maintained for longer than 12 months in the subject.

In some embodiments, the method provided herein comprises assessing duration of response in the subject.

In some embodiments, the method provided herein comprises determining that the subject is likely to have a duration of response of more than 12 months if the MRD negative status is maintained for longer than 6 months in the subject.

In some embodiments, the method provided herein comprises determining that the subject is likely to have a duration of response of more than 24 months if the MRD negative status is maintained for longer than 12 months in the subject.

### 4. BRIEF DESCRIPTION OF THE DRAWINGS

FIG. 1 shows the expression of BCMA antigen on the surface of GC, memory and plasmablast cells in the lymph node, long-lived plasma cells in the bone marrow LN and MALT, and on multiple myeloma cells. BAFF-R antigen is not expressed on plasmablast cells, long-lived plasma cells, or multiple myeloma cells. TACI is expressed on memory and plasmablast cells, long-lived plasma cells, and multiple myeloma cells. CD138 is expressed only on long-lived plasma cells and multiple myeloma cells.

FIG. 2 shows the design of the ciltacabtagene autoleucel CAR. Ciltacabtagene autoleucel comprises two VHH domains, as opposed to a single VL domain and a single VH domain found on various other CARs. Ciltacabtagene autoleucel comprises intracellular CD137 and human CD3 zeta domains.

FIG. 3 shows a schematic for preparing virus encoding ciltacabtagene autoleucel CAR, transduction of the virus into a T cell from the patient, and then preparation of CAR T cells expressing ciltacabtagene autoleucel.

FIG. 4 summarizes demographic and disease characteristics of the patient population in the Phase 1b portion of the study.

FIG. 5 shows a schematic of study design for ciltacabtagene autoleucel CAR T-cells. The patient population includes those with relapsed or Refractory Multiple Myeloma, with 3 prior lines or double refractory to PI/IMiD and prior PI, IMiD, anti-CD38 exposure. A primary objective is safety and establishment of RP2D, such as studying incidence and severity of adverse events (Phase 1b). Another primary objective is efficacy: ORR-PR or better as defined by IMWG (Phase 2). The following are secondary objectives: Incidence and severity of adverse events (Phase 2), and any further efficacy characterization.

FIG. 6 shows expansion and persistence of cilta-cel as measured by blood concentration.

FIG. 7 shows progression-free survival outcomes for patient subgroups, based on MRD negativity status. MRD negative<6 months: patients who achieved MRD negativity but became MRD positive less than 6 months later. MRD negative≥6: patients who achieved MRD negativity but became MRD positive more than 6 but less than 12 months later. MRD negative≥12 months: patients who achieved MRD negativity and remained MRD negative for 12 months or longer. MRD positive: patients who did not achieve MRD negativity at any time point. The top plot shows censored observations in the corresponding patient subgroup. The x-axis shows the time, in months, that patients in each subgroup have remained alive and progression-free. The y-axis represents the percent of patients in each subgroup who are alive and progression-free after a given length of time, indicated by the x-axis. The table below the plot shows the number of patients in each subgroup who are still progression-free and alive at each time point, aligned with 3-month intervals shown on the x-axis of the plot.

FIG. 8 shows duration of response for patient subgroups, based on MRD negativity status. MRD negative<6 months: patients who achieved MRD negativity but became MRD positive less than 6 months later. MRD negative≥6 and <12 months: patients who achieved MRD negativity but became MRD positive more than 6 but less than 12 months later. MRD negative≥12 months: patients who achieved MRD negativity and remained MRD negative for 12 months or longer. MRD positive: patients who did not achieve MRD negativity at any time point. The top plot represents censored observations in the corresponding patient subgroup. The x-axis shows the time, in months, that patients who responded (responders) in each subgroup have remained alive and progression-free, i.e., those who have continued to respond. The y-axis represents the percent of responders in each subgroup who are alive and progression-free after a given length of time, indicated by the x-axis. The table below the plot shows the number of responders in each subgroup who are still responding at each time point, aligned with 3-month intervals shown on the x-axis of the plot.

FIG. 9 shows key patient and disease characteristics in these different MRD negativity groups. ^{a}≥1 PI, ≥1 IMiD, and 1 anti-CD38 antibody. ^{b}₂ PIs, 2 IMiDs, and 1 anti-CD38 antibody. ECOG, Eastern Cooperative Oncology Group; IMiD, immunomodulatory drug; PI, proteasome inhibitor.

FIG. 10 shows responses in MRD positive patients. Patients who did not achieve MRD negativity at any time point were considered to be MRD positive (n=5). The y-axis lists deidentified patient numbers. The x-axis shows the time, in months.

FIG. 11 shows response to cilta-cel in MRD subgroups. All patients with sustained MRD negativity for 6 months achieved sCR. ^{a}ORR=sCR+CR+VGPR+PR; may not sum appropriately due to rounding. DOR, duration of response; MRD, minimal residual disease; ORR, overall response rate; PR, partial response; sCR, sustained complete response; VGPR, very good partial response.

FIG. 12 shows progression-free survival outcomes for patient subgroups, based on MRD negativity status. MRD negative<6 months: patients who achieved MRD negativity but became MRD positive less than 6 months later. MRD negative≥6: patients who achieved MRD negativity but became MRD positive more than 6 but less than 12 months later. MRD negative≥12 months: patients who achieved MRD negativity and remained MRD negative for 12 months or longer.

### 5. DETAILED DESCRIPTION

The present disclosure is based, in part, on the surprising finding of the correlation between the time length a subject maintains minimal residual disease (MRD) negative status and responsiveness (as measured by various approaches) of the subject to the treatment with engineered T cells expressing bivalent BCMA-targeting CAR. Thus, the length a subject maintains MRD negative status may be used to determine responsiveness of the subject to the treatment with engineered T cells expressing bivalent BCMA-targeting CAR (such as “Ciltacabtagene autoleucel” or “cilta-cel”).

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

“Single domain antibody” or “sdAb” as used herein refers to a single monomeric variable antibody domain and which is capable of antigen binding (e.g., single domain antibodies that bind to BCMA). Single domain antibodies include VHH domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca and guanaco, as described herein. Other species besides Camelidae may produce heavy chain antibodies naturally devoid of light chain; VHHs derived from such other species are within the scope of the disclosure. In some embodiments, the single domain antibody (e.g., VHH) provided herein has a structure of FR1-CDR1-FR2-CDR2-FR3-CDR3-FR4. Single domain antibodies may be genetically fused or chemically conjugated to another molecule (e.g., an agent) as described herein. Single domain antibodies may be part of a bigger binding molecule (e.g., a multispecific antibody or a chimeric antigen receptor).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet©, Biacore©, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis or RIA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D}) of less than or equal to 1 M, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise a single domain antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1: 755-68 (2006)). Also available for the preparation of human monoclonal antibodies are methods described in Cole et al., Monoclonal Antibodies and Cancer Therapy 77 (1985); Boerner et al., J. Immunol. 147(1):86-95 (1991); and van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6(5):561-66 (1995); Bruggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid isomerization or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System© (Lafranc et al., Dev. Comp. Immunol. 27(1):55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in table below.

Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105-L117 |
| | | | L89--L97 | | |
| CDR H1 | H31--H35B | H26--H35B | H26--H32 . . . 34 | H30--H35B | H27--H38 |
| | (Kabat | | | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 or | H93--H101 | H105-H117 |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR as set forth in a specific VH or VHH” includes any CDR1 as defined by the exemplary CDR numbering systems described above, but is not limited thereby. Once a variable region (e.g., a VHH, VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies (e.g., single domain antibodies), diabodies, linear antibodies, and bispecific antibodies. FR residues are those variable domain residues other than the hypervariable region residues or CDR residues.

As used herein, an “epitope” is a term in the art and refers to a localized region of an antigen to which a binding molecule (e.g., an antibody comprising a single domain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “specificity” refers to selective recognition of an antigen binding protein (such as a CAR or an sdAb) for a particular epitope of an antigen. Natural antibodies, for example, are monospecific. The term “multispecific” as used herein denotes that an antigen binding protein (such as a CAR or an sdAb) has two or more antigen-binding sites of which at least two bind different antigens. “Bispecific” as used herein denotes that an antigen binding protein (such as a CAR or an sdAb) has two different antigen-binding specificities. The term “monospecific” CAR as used herein denotes an antigen binding protein (such as a CAR or an sdAb) that has one or more binding sites each of which bind the same antigen.

The term “valent” as used herein denotes the presence of a specified number of binding sites in an antigen binding protein (such as a CAR or an sdAb). A natural antibody for example or a full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein (such as a CAR or an sdAb).

“Chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” In some embodiments, the CAR comprises an extracellular antigen binding domain specific for one or more antigens (such as tumor antigens), a transmembrane domain, and an intracellular signaling domain of a T cell and/or other receptors. “CAR-T cell” refers to a T cell that expresses a CAR.

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs”, which refer to T cells that suppress an abnormal or excessive immune response and play a role in immune tolerance. Tregs are typically transcription factor Foxp3-positive CD4+ T cells and can also include transcription factor Foxp3-negative regulatory T cells that are IL-10-producing CD4+ T cells.

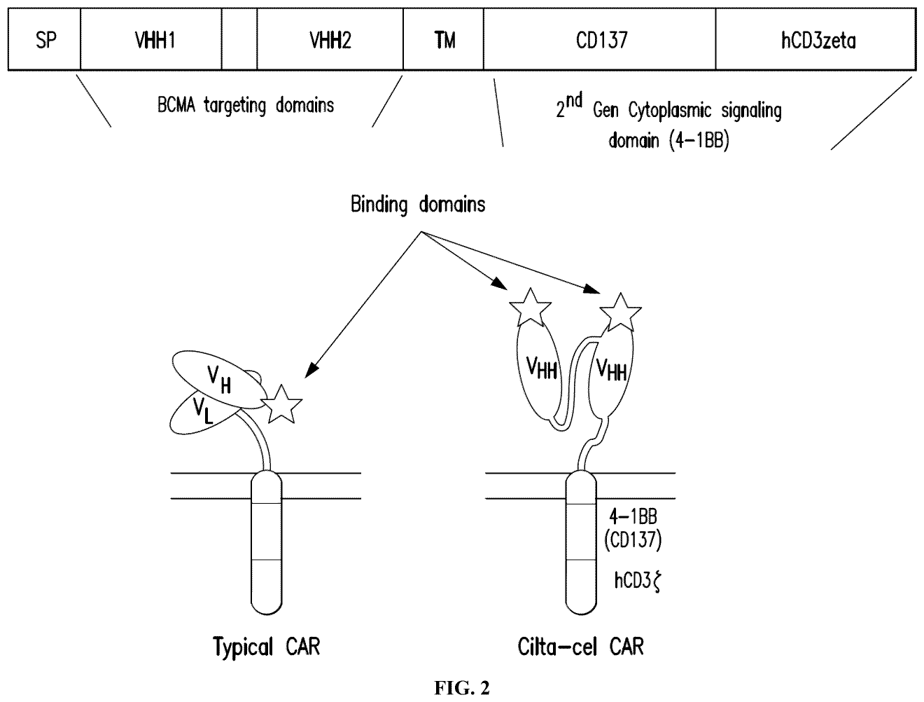

The term “Ciltacabtagene autoleucel” or “cilta-cel” refers to a chimeric antigen receptor T cell (CAR-T) therapy comprising two B-cell maturation antigen (BCMA)-targeting VHH domains designed to confer avidity for BCMA. Cilta-cel can comprise T lymphocytes transduced with the ciltacabtagene autoleucel CAR, a CAR encoded by a lentiviral vector. The CAR targets the human B cell maturation antigen (anti-BCMA CAR). A diagram of the lentiviral vector encoding cilta-cel CAR is provided in FIG. 2. The amino acid sequence of the cilta-cel CAR is the amino acid sequence of SEQ ID NO: 17.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding a single domain antibody or an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding a CAR or an sdAb described herein is a nucleic acid molecule that is identified and separated from at least one contaminant nucleic acid molecule with which it is ordinarily associated in the environment in which it was produced.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “autologous” is meant to refer to any material derived from the same individual to whom it is later to be re-introduced into the individual.

“Allogeneic” refers to a graft derived from a different individual of the same species.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

The term “effective amount” or “therapeutically effective amount” as used herein refers to the amount of a single domain antibody or a therapeutic molecule comprising an agent and the single domain antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The term “line of therapy,” as used in connection with methods of treatment herein, refers to one or more cycles of a planned treatment program, which may have consisted of one or more planned cycles of single-agent therapy or combination therapy, as well as a sequence of treatments administered in a planned manner. For example, a planned treatment approach of induction therapy followed by autologous stem cell transplantation followed by maintenance is one line of therapy. A new line of therapy is considered to have started when a planned course of therapy has been modified to include other treatment agents or medicaments (alone or in combination) as a result of disease progression, relapse, or toxicity. A new line of therapy is also considered to have started when a planned period of observation off therapy had been interrupted by a need for additional treatment for the disease.

The term “refractory,” as used in connection to treatment with a particular treatment agent or medicament or line of therapy herein, refers to diseases or disease subjects that fail to respond to said treatment agent or medicament or line of therapy. The phrase “refractory myeloma” refers to multiple myeloma that is nonresponsive while on primary or salvage therapy or that has progressed within 60 days of last therapy.

The phrase “nonresponsive disease” refers to either failure to achieve minimal response or to development of progressive disease while on therapy.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in cancer cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30, or more times (e.g., 500, 1000 times) (including all integers and decimal points in-between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in-between and above 1, e.g., 1.5, 1.6, 1.7. 1.8, etc.) the response (reference response) produced by vehicle, a control composition, or the response in a particular cell lineage.

The terms “prevent,” “preventing,” and “prevention” refer to reducing the likelihood of the onset (or recurrence) of a disease, disorder, condition, or associated symptom(s) (e.g., diabetes or a cancer).

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

“B cell associated disease or disorder” as used herein refers to a disease or disorder mediated by B cells or conferred by abnormal B cell functions (such as dysregulation of B-cell function). “B cell associated disease or disorder” as used herein includes but not limited to a B cell malignancy such as a B cell leukemia or B cell lymphoma. It also includes marginal zone lymphoma (e.g., splenic marginal zone lymphoma), diffuse large B cell lymphoma (DLBCL), mantle cell lymphoma (MCL), primary central nervous system (CNS) lymphoma, primary mediastinal B cell lymphoma (PMBL), small lymphocytic lymphoma (SLL), B cell prolymphocytic leukemia (B-PLL), follicular lymphoma (FL), burkitt lymphoma, primary intraocular lymphoma, chronic lymphocytic leukemia (CLL), acute lymphoblastic leukemia (ALL), hairy cell leukemia (HCL), precursor B lymphoblastic leukemia, non-hodgkin lymphoma (NHL), high-grade B-cell lymphoma (HGBL), and multiple myeloma (MM). “B cell associated disease or disorder” also includes certain autoimmune and/or inflammatory disease, such as those associated with inappropriate or enhanced B cell numbers and/or activation.

“BCMA associated disease or disorder” as used herein refers to a disease or disorder that comprises a cell or tissue in which BCMA is expressed or overexpressed. In some embodiments, BCMA associated disease or disorder comprises a cell on which BCMA is abnormally expressed. In other embodiments, BCMA associated disease or disorder comprises a cell in or on which BCMA is deficient.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2. Methods for Assessing Responsiveness to Treatment with CAR-T Cells

In one aspect, provided herein is a method for assessing responsiveness of a subject to a treatment comprising T cells expressing a bivalent BCMA-targeting chimeric antigen receptor (CAR) based on time length the subject maintains minimal residual disease (MRD) negative status. In some embodiments, the method comprises administering to the subject the T cells; measuring time length the subject maintains MRD negative status; and assessing the responsiveness of the subject to the treatment based on the time length the subject maintains MRD negative status.

Methods for determining MRD status and measuring the time length a subject maintains MRD negative status are known in the art, and any of those methods may be used in the present methods.

In some embodiments, the method comprises obtaining a sample from bone marrow (such as bone marrow aspirate or biopsy) from the subject for assessing MRD status. In certain embodiments, clinical staging (morphology, cytogenetics, and immunohistochemistry or immunofluorescence or flow cytometry) may be done. In certain embodiments, a portion of the bone marrow aspirate may be immunophenotyped and monitored for BCMA, checkpoint ligand expression in CD138-positive multiple myeloma cells, and checkpoint expression on T cells. In certain embodiments, MRD may be monitored in subjects using next generation sequencing (NGS) of bone marrow aspirate DNA. The NGS of bone marrow aspirate DNA is known to one of ordinary skill in the art. In certain embodiments, the NGS is performed via clonoSEQ. In certain embodiments, baseline bone marrow aspirates may be used to define the myeloma clones, and post-treatment samples may be used to evaluate MRD negativity. In certain embodiments, the MRD negativity status may be based on samples that are evaluable. In certain embodiments, evaluable samples are those that passed one or more of, or all of, calibration, quality control, and sufficiency of cells evaluable at a particular sensitivity level. In some embodiments, the sensitivity level is about 10⁻⁶. In certain embodiments, the sensitivity level is about 10⁻⁵. In certain embodiments, the sensitivity level is about 10⁻⁴. In certain embodiments, the sensitivity level is about 10⁻³.

In some embodiments, the method comprises assessing the responsiveness of the subject to the treatment based on that the MRD negative status is shorter than 6 months. In some embodiments, the method comprises assessing the responsiveness of the subject to the treatment based on that the MRD negative status is at least 6 months and shorter than 12 months. In other embodiments, the method comprises assessing the responsiveness of the subject to the treatment based on that the MRD negative status is at least 12 months.

In some embodiments, the method comprises assessing the likelihood of the subject to have complete response (CR), partial response (PR), stringent CR (sCR), or very good PR (VGPR). In some embodiments, the method comprises determining that the subject is likely to have CR, PR, sCR, or VGPR if the MRD negative status is maintained for shorter than 6 months in the subject. In some embodiments, the method comprises determining that the subject is likely to have CR or sCR if the MRD negative status is maintained for longer than 6 months in the subject.

In certain embodiments, a subject's response to the treatment is assessed using the International Myeloma Working Group (IMWG)-based response criteria, which are summarized in Table 6. In certain embodiments, the response may be classified as a stringent complete response (sCR). In certain embodiments, the response may be classified as a complete response (CR), which is worse than a stringent complete response (sCR). In certain embodiments, the response may be classified as a very good partial response (VGPR), which is worse than a complete response (CR). In certain embodiments, the response may be classified as a partial response (PR), which is worse than a very good partial response (VGPR). In certain embodiments, the response may be classified as a minimal response (MR), which is worse than a partial response (PR). In certain embodiments, the response may be classified as a stable disease (SD), which is worse than a minimal response (MR). In certain embodiments, the response may be classified as a progressive disease (PD), which is worse than a stable disease.

In certain embodiments, the tests used to assess International Myeloma Working Group (IMWG)-based response criteria are Myeloma protein (M-protein) measurements in serum and urine, serum calcium corrected for albumin, bone marrow examination, skeletal survey and documentation of extramedullary plasmacytomas.

Non-limiting examples of tests for M-protein measurement in blood and urine are known to one of ordinary skill in the art and comprise serum quantitative Ig, serum protein electrophoresis (SPEP), serum immunofixation electrophoresis, serum FLC assay, 24-hour urine M-protein quantitation by electrophoresis (UPEP), urine immunofixation electrophoresis, and serum β2-microglobulin.

Calculating serum calcium corrected for albumin in blood samples for detection of hypercalcemia is known to one of ordinary skill in the art. Without wishing to be bound by theory, calcium binds to albumin and only the unbound (free) calcium is biologically active; therefore, the serum calcium level must be adjusted for abnormal albumin levels (“corrected serum calcium”).

In certain embodiments, a skeletal survey of any one of, or all of, the skull, the entire vertebral column, the pelvis, the chest, the humeri, the femora, and any other bones, may be performed and evaluated by either roentgenography (“X-rays”) or low-dose computed tomography (CT) diagnostic quality scans without the use of IV contras, both of which are known to one of ordinary skill in the art. In certain embodiments, following T cell administration and before disease progression is confirmed, X-rays or CT scans may be performed locally, whenever clinically indicated based on symptoms, to document response or progression. In certain embodiments, magnetic resonance imaging (MRI) may be used for evaluating bone disease but does not replace a skeletal survey. MRI is known to one of ordinary skill in the art. In certain embodiments, if a radionuclide bone scan is used at screening, in addition to the complete skeletal survey, both methods may be used to document disease status. Radionuclide bone scans are known to one of ordinary skill in the art. In certain embodiments, the radionuclide bone scan and complete skeletal survey may be performed at the same time. In certain embodiments, a radionuclide bone scan may not replace a complete skeletal survey. In certain embodiments, if a subject presents with disease progression manifested by symptoms of pain due to bone changes, then disease progression may be documented by skeletal survey or other radiographs, depending on the symptoms that the subject experiences.

In certain embodiments, extramedullary plasmacytomas may be documented by clinical examination or MRI. In certain embodiments, if there was no contraindication to the use of IV contrast, extramedullary plasmacytomas may be documented by CT scan. In certain embodiments, extramedullary plasmacytomas may be documented by a fusion of positron emission tomography (PET) and CT scans if the CT component is of sufficient diagnostic quality. In certain embodiments, assessment of measurable sites of extramedullary disease may be performed, measured, or evaluated locally every 4 weeks for subjects until development of confirmed CR or confirmed disease progression. In certain embodiments, evaluation of extramedullary plasmacytomas may be done every 12 weeks.

In certain embodiments, to qualify for VGPR or PR or MR, the sum of products of the perpendicular diameters of the existing extramedullary plasmacytomas may have decreased by over 90% or at least 50%, respectively. In certain embodiments, to qualify for disease progression, either the sum of products of the perpendicular diameters of the existing extramedullary plasmacytomas must have increased by at least 50%, or the longest diameter of previous lesion>1 cm in short axis must have increased at least 50%, or a new plasmacytoma must have developed. In certain embodiments, to qualify for disease progression when not all existing extramedullary plasmacytomas are reported, the sum of products of the perpendicular diameters of the reported plasmacytomas had increased by at least 50%. In certain embodiments, if the study treatment interferes with the immunofixation assay, CR may be defined as the disappearance of the original M-protein associated with multiple myeloma on immunofixation.

In other embodiments, the method comprises assessing the likelihood of the subject to have progression-free survival, e.g., at a time point after the treatment. In some embodiments, the method comprises determining that the subject is likely to have progression-free survival for at least 12 months post the treatment if the MRD negative status is maintained for longer than 6 months in the subject. In some embodiments, the method comprises determining that the subject is likely to have progression-free survival for at least 24 months post the treatment if the MRD negative status is maintained for longer than 12 months in the subject.

In yet other embodiments, the method comprises assessing duration of response in the subject. In some embodiments, the method comprises determining that the subject is likely to have a duration of response of more than 12 months if the MRD negative status is maintained for longer than 6 months in the subject. In other embodiments, the method comprises determining that the subject is likely to have a duration of response of more than 24 months if the MRD negative status is maintained for longer than 12 months in the subject.

5.3. Bi-valent BCMA-Targeting CARs

The T cells provided in the present methods express a bivalent BCMA-targeting CAR comprising an extracellular antigen binding domain comprising a first VHH domain and a second VHH domain, a transmembrane domain, and an intracellular signaling domain, wherein the first VHH domain comprising a CDR1, a CDR2, and a CDR3 as set forth in the VHH domain comprising the amino acid sequence of SEQ ID NO: 2, and the second VHH domain comprising a CDR1, a CDR2, and a CDR3 as set forth in the VHH domain comprising the amino acid sequence of SEQ ID NO: 4. In some embodiments, the CDR1, CDR2 or CDR3 are determined according to the Kabat numbering scheme, the IMGT numbering scheme, the AbM numbering scheme, the Chothia numbering scheme, the Contact numbering scheme, or a combination thereof. In some specific embodiments, the first VHH domain comprises a CDR1 comprising the amino acid sequence of SEQ ID NO: 18, a CDR2 comprising the amino acid sequence of SEQ ID NO: 19, a CDR3 comprising the amino acid sequence of SEQ ID NO: 20; and the second VHH domain comprises a CDR1 comprising the amino acid sequence of SEQ ID NO: 21, a CDR2 comprising the amino acid sequence of SEQ ID NO: 22, and a CDR3 comprising the amino acid sequence of SEQ ID NO: 23. In some embodiments, the first VHH domain comprises the amino acid sequence of SEQ ID NO: 2 and the second VHH domain comprises the amino acid sequence of SEQ ID NO: 4. In some embodiments, the first VHH domain is at the N-terminus of the second VHH domain. In other embodiments, the first VHH domain is at the C-terminus of the second VHH domain. In some embodiments, the first VHH domain is linked to the second VHH domain via a linker comprising the amino acid sequence of SEQ ID NO: 3.

The first VHH domain and the second VHH domain may be linked via a peptide linker. Different domains of the CARs may also be fused to each other via peptide linkers.

Each peptide linker in a CAR may have the same or different length and/or sequence depending on the structural and/or functional features of the antibodies and/or the various domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. For example, longer peptide linkers may be selected to ensure that two adjacent domains do not sterically interfere with one another. In some embodiments, a short peptide linker may be disposed between the transmembrane domain and the intracellular signaling domain of a CAR. In some embodiment, a peptide linker comprises flexible residues (such as glycine and serine) so that the adjacent domains are free to move relative to each other. For example, a glycine-serine doublet can be a suitable peptide linker.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)n, glycine-serine polymers, glycine-alanine polymers, alanine-serine polymers, and other flexible linkers known in the art. Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the CARs provided herein, the disclosure of each of which is incorporated herein by reference.

In some specific embodiments, the peptide linker connecting the first VHH domain and the second VHH domain comprises the amino acid sequence of SEQ ID NO: 3.

The CARs of the present disclosure comprise a transmembrane domain that can be directly or indirectly fused to the extracellular antigen binding domain. The transmembrane domain may be derived either from a natural or from a synthetic source. As used herein, a “transmembrane domain” refers to any protein structure that is thermodynamically stable in a cell membrane, preferably a eukaryotic cell membrane. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl la, CD18), ICOS (CD278), 4-1BB (CD137), GITR, CD40, BAFFR, HVEM (LIGHTR), SLAMF7, NKp80 (KLRFI), CD160, Claudin-6, IL-2R beta, IL-2R gamma, IL-7R a, ITGA1, VLA1, CD49a, ITGA4, IA4, CD49D, ITGA6, VLA-6, CD49f, ITGAD, CDl id, ITGAE, CD103, ITGAL, CDl la, LFA-1, ITGAM, CDl lb, ITGAX, CDl 1c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain is derived from a molecule selected from the group consisting of CD8a, CD4, CD28, CD137, CD80, CD86, CD152 and PD1.

In some specific embodiments, the transmembrane domain is derived from CD8a. In some embodiments, the transmembrane domain is a transmembrane domain of CD8a comprising the amino acid sequence of SEQ ID NO: 6.

Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the CAR provided herein comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

The CARs of the present disclosure comprise an intracellular signaling domain. The intracellular signaling domain is responsible for activation of at least one of the normal effector functions of the immune effector cell expressing the CARs. The term “effector function” refers to a specialized function of a cell. Effector function of a T cell, for example, may be cytolytic activity or helper activity including the secretion of cytokines. Thus the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal.

In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3ζ, FcR gamma (FCER1G), FcR beta (Fc Epsilon Rib), CD3 gamma, CD3 delta, CD3 epsilon, CD5, CD22, CD79a, CD79b, and CD66d.

In some embodiments, the primary intracellular signaling domain is derived from CD3ζ. In some embodiments, the intracellular signaling domain consists of the cytoplasmic signaling domain of CD3ζ. In some embodiments, the primary intracellular signaling domain is a cytoplasmic signaling domain of wild-type CD3ζ. In some embodiments, the primary intracellular signaling domain of CD3ζ comprises the amino acid sequence of SEQ ID NO: 8.

Many immune effector cells require co-stimulation, in addition to stimulation of an antigen-specific signal, to promote cell proliferation, differentiation and survival, as well as to activate effector functions of the cell. In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3ζ) and one or more co-stimulatory signaling domains. In some embodiments, the one or more co-stimulatory signaling domains and the primary intracellular signaling domain (such as cytoplasmic signaling domain of CD3ζ) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3ζ). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C.

In some embodiments, the one or more co-stimulatory signaling domains are selected from the group consisting of CD27, CD28, CD137, OX40, CD30, CD40, CD3, lymphocyte function-associated antigen-1(LFA-1), CD2, CD7, LIGHT, NKG2C, B7-H3 and ligands that specially bind to CD83.

In some embodiments, the intracellular signaling domain in the CAR of the present disclosure comprises a co-stimulatory signaling domain derived from CD137 (i.e., 4-1B). In some embodiments, the intracellular signaling domain comprises a cytoplasmic signaling domain of CD3ζ and a co-stimulatory signaling domain of CD137. In some embodiments, the intracellular signaling domain comprises a co-stimulatory signaling domain of CD137 comprising the amino acid sequence of SEQ ID NO: 7.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

The CARs of the present disclosure may comprise a hinge domain that is located between the extracellular antigen binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is derived from CD8a. In some embodiments, the hinge domain is a portion of the hinge domain of CD8a, e.g., a fragment containing at least 15 (e.g., 20, 25, 30, 35, or 40) consecutive amino acids of the hinge domain of CD8a. In some embodiments, the hinge domain of CD8a comprises the amino acid sequence of SEQ ID NO: 5.

Hinge domains of antibodies, such as an IgG, IgA, IgM, IgE, or IgD antibodies, are also compatible for use in the pH-dependent chimeric receptor systems described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (G×S)n linker, wherein x and n, independently can be an integer between 3 and 12, including 3, 4, 5, 6, 7, 8, 9, 10, 11, 12, or more.

The CARs of the present disclosure may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8a, GM-CSF receptor α, and IgG1 heavy chain. In some embodiments, the signal peptide is derived from CD8α. In some embodiments, the signal peptide of CD8a comprises the amino acid sequence of SEQ ID NO: 1.

In a specific embodiment, the CAR provided herein comprises the amino acid sequence of SEQ ID NO: 17

### 5.4. Engineered Immune Effector Cells

The immune effector cells (such as T cells) provided herein express comprising the CARs described herein (see Section 5.3 above). “Immune effector cells” are immune cells that can perform immune effector functions. In some embodiments, the immune effector cells express at least FcγRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils.

In some embodiments, the immune effector cells are T cells. In some embodiments, the T cells are CD4+/CD8−, CD4−/CD8+, CD4+/CD8+, CD4−/CD8−, or combinations thereof. In some embodiments, the T cells produce IL-2, TFN, and/or TNF upon expressing the CAR and binding to the target cells, such as GPC3+ tumor cells. In some embodiments, the CD8+ T cells lyse antigen-specific target cells upon expressing the CAR and binding to the target cells.

The engineered immune effector cells are prepared by introducing the CARs into the immune effector cells, such as T cells. In some embodiments, the CAR is introduced to the immune effector cells by transfecting any one of the isolated nucleic acids or any one of the vectors described above. In some embodiments, the CAR is introduced to the immune effector cells by inserting proteins into the cell membrane while passing cells through a microfluidic system, such as CELL SQUEEZE© (see, e.g., U.S. Patent Application Publication No. 20140287509).

Methods of introducing vectors or isolated nucleic acids into a mammalian cell are known in the art. The vectors described can be transferred into an immune effector cell by physical, chemical, or biological methods.

Physical methods for introducing the vector into an immune effector cell include calcium phosphate precipitation, lipofection, particle bombardment, microinjection, electroporation, and the like. Methods for producing cells comprising vectors and/or exogenous nucleic acids are well-known in the art. See, e.g., Sambrook et al. (2001) Molecular Cloning: A Laboratory Manual, Cold Spring Harbor Laboratory, New York. In some embodiments, the vector is introduced into the cell by electroporation.

Biological methods for introducing the vector into an immune effector cell include the use of DNA and RNA vectors. Viral vectors have become the most widely used method for inserting genes into mammalian, e.g., human cells.

Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, RNA molecules encoding any of the CARs described herein may be prepared by a conventional method (e.g., in vitro transcription) and then introduced into the immune effector cells via known methods such as mRNA electroporation. See, e.g., Rabinovich et al., Human Gene Therapy 17:1027-1035 (2006).

In some embodiments, the transduced or transfected immune effector cell is propagated ex vivo after introduction of the vector or isolated nucleic acid. In some embodiments, the transduced or transfected immune effector cell is cultured to propagate for at least about any of 1 day, 2 days, 3 days, 4 days, 5 days, 6 days, 7 days, 10 days, 12 days, or 14 days. In some embodiments, the transduced or transfected immune effector cell is further evaluated or screened to select the engineered mammalian cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)). Suitable expression systems are well known and may be prepared using known techniques or obtained commercially.

Other methods to confirm the presence of the nucleic acid encoding the CARs in the engineered immune effector cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

In some embodiments, prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art, may be used. In some embodiments, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Initial activation steps in the absence of calcium may lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, in some embodiments, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-CD25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations may result in increased cell yield, cell activation, and cell expansion. Further, use of high cell concentrations may allow more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. In some embodiments, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/mL. In some embodiments, the concentration used can be from about 1×10⁵/mL to 1×10⁶/mL, and any integer value in between.

In some embodiments, the cells may be incubated on a rotator for varying lengths of time at varying speeds at either 2-10° C., or at room temperature.

T cells for stimulation can also be frozen after a washing step. Without being bound by theory, the freeze and subsequent thaw step may provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% dextran 40 and 5% dextrose, 20% human serum albumin and 7.5% DMSO, or 31.25% plasmalyte-A, 31.25% dextrose 5%, 0.45% NaCl, 10% dextran 40 and 5% dextrose, 20% human serum albumin, and 7.5% DMSO or other suitable cell freezing media containing for example, Hespan and PlasmaLyte A. The cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

Also contemplated in the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment, a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815 (1991); Henderson et al., Immun 73:316-321 (1991); Bierer et al., Curr. Opin. Immun. 5:763-773 (1993)). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH.

In some embodiments, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In some embodiments, prior to or after genetic modification of the T cells with the CARs described herein, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD3 antibody include UCHT1, OKT3, HIT3a (BioLegend, San Diego, US) can be used as can other methods commonly known in the art (Graves J, et al., J. Immunol. 146:2102 (1991); Li B, et al., Immunology 116:487 (2005); Rivollier A, et al., Blood 104:4029 (2004)). Examples of an anti-CD28 antibody include 9.3, B-T3, XR-CD28 (Diaclone, Besancon, France) can be used as can other methods commonly known in the art (Berg et al., Transplant Proc. 30(8):3975-3977 (1998); Haanen et al., J. Exp. Med. 190(9):13191328 (1999); Garland et al., J. Immunol Meth. 227(1-2):53-63 (1999)).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in certain embodiments in the present disclosure.

In some embodiments, the T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment, the cells (for example, 10⁴ to 4×10⁸ T cells) and beads (for example, anti-CD3/CD28 MACSiBead particles at a recommended titer of 1:100) are combined in a buffer, preferably PBS (without divalent cations such as, calcium and magnesium). Those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations may result in increased cell yield, cell activation, and cell expansion. Further, use of high cell concentrations may allow more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments, the mixture may be cultured for several hours (about 3 hours) to about 14 days or any hourly integer value in between. In another embodiment, the mixture may be cultured for 21 days. In one embodiment, the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-7, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

In some embodiments, provided herein are dosage forms comprising 3.0×10⁷ to 1.0×10⁸ of the present CAR-T cells. In some embodiments, there are provided dosage forms comprising 3.0×10⁷ to 1.0×10⁸ of the present CAR-T cells. In certain embodiments, the dosage form comprises 3.0×10⁷ to 4.0×10⁷ of the CAR-T cells. In certain embodiments, the dosage form comprises 3.5×10⁷ to 4.5×10⁷ of the CAR-T cells. In certain embodiments, the dosage form comprises 4.0×10⁷ to 5.0×10⁷ of the CAR-T cells. In certain embodiments, the dosage form comprises 4.5×10⁷ to 5.5×10⁷ of the CAR-T cells. In certain embodiments, the dosage form comprises 5.0×10⁷ to 6.0×10⁷ of the CAR-T cells. In certain embodiments, the dosage form comprises 5.5×10⁷ to 6.5×10⁷ of the CAR-T cells. In certain embodiments, the dosage form comprises 6.0×10⁷ to 7.0×10⁷ of the CAR-T cells. In certain embodiments, the dosage form comprises 6.5×10⁷ to 7.5×10⁷ of the CAR-T cells. In certain embodiments, the dosage form comprises 7.0×10⁷ to 8.0×10⁷ of the CAR-T cells. In certain embodiments, the dosage form comprises 7.5×10⁷ to 8.5×10⁷ of the CAR-T cells. In certain embodiments, the dosage form comprises 8.0×10⁷ to 9.0×10⁷ of the CAR-T cells. In certain embodiments, the dosage form comprises 8.5×10⁷ to 9.5×10⁷ of the CAR-T cells. In certain embodiments, the dosage form comprises 9.0×10⁷ to 1.0×10⁸ of the CAR-T cells.

In some embodiments, the cell population of the CAR-T dosage forms described herein comprise a T cell or population of T cells, e.g., at various stages of differentiation. Stages of T cell differentiation include naïve T cells, stem central memory T cells, central memory T cells, effector memory T cells, and terminal effector T cells, from least to most differentiated. After antigen exposure, naïve T cells proliferate and differentiate into memory T cells, e.g., stem central memory T cells and central memory T cells, which then differentiate into effector memory T cells. Upon receiving appropriate T cell receptor, costimulatory, and inflammatory signals, memory T cells further differentiate into terminal effector T cells. See, e.g., Restifo. Blood. 124.4(2014):476-77; and Joshi et al. J. Immunol. 180.3(2008):1309-15.

Naïve T cells can have the following expression pattern of cell surface markers: CCR7+, CD62L+, CD45RO−, CD95−. Stem central memory T cells (Tscm) can have the following expression pattern of cell surface markers: CCR7+, CD62L+, CD45RO−, CD95+. Central memory T cells (Tcm) can have the following expression pattern of cell surface markers: CCR7+, CD62L+, CD45RO+, CD95+. Effector memory T cells (Tem) can have the following expression pattern of cell surface markers: CCR7−, CD62L−, CD45RO+, CD95+. Terminal effector T cells (Teff) can have the following expression pattern of cell surface markers: CCR7−, CD62L−, CD45RO−, CD95+. See, e.g., Gattinoni et al. Nat. Med. 17(2011):1290-7; and Flynn et al. Clin. Translat. Immunol. 3(2014):e20.

### 5.5. Vectors

Polynucleotide sequences encoding the CARs described in the present application can be obtained using standard recombinant techniques. Desired polynucleotide sequences may be isolated and sequenced from antibody producing cells such as hybridoma cells. Alternatively, polynucleotides can be synthesized using nucleotide synthesizers or PCR techniques.

The disclosure also provides a vector comprising the nucleic acid sequence encoding the CARs disclosed herein. The vector can be, for example, a plasmid, a cosmid, a viral vector (e.g., retroviral or adenoviral), or a phage. Suitable vectors and methods of vector preparation are well known in the art (see, e.g., Sambrook et al. and Ausubel et al.).

In addition to the nucleic acid sequences encoding the CARs disclosed herein, the vector preferably comprises expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, Calif. (1990).

In some embodiments, the vector comprises a promoter. A large number of promoters recognized by a variety of potential host cells are well known. The selected promoter can be operably linked to cistron DNA encoding the CARs disclosed herein by removing the promoter from the source DNA via restriction enzyme digestion and inserting the isolated promoter sequence into the vector of the present application. A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, CA), LACSWITCH™ System (Stratagene, San Diego, CA), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol, 308: 123-144 (2005)).

In some embodiments, the vector comprises an “enhancer”. The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (e.g., from depositories such as the ATCC as well as other commercial or individual sources). A number of polynucleotides comprising promoters (such as the commonly used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus. Such Ig enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

In some embodiments, the vector comprises a “selectable marker gene.” The term “selectable marker gene”, as used herein, refers to a nucleic acid sequence that allows cells expressing the nucleic acid sequence to be specifically selected for or against, in the presence of a corresponding selective agent. Suitable selectable marker genes are known in the art and described in, e.g., International Patent Application Publication Nos. WO 1992/08796 and WO 1994/28143; Wigler et al., Proc. Natl. Acad. Sci. USA, 77: 3567 (1980); O'Hare et al., Proc. Natl. Acad. Sci. USA, 78: 1527 (1981); Mulligan & Berg, Proc. Natl. Acad. Sci. USA, 78: 2072 (1981); Colberre-Garapin et al., J. Mol. Biol., 150: 1 (1981); Santerre et al., Gene, 30: 147 (1984); Kent et al., Science, 237: 901-903 (1987); Wigler et al., Cell, IP. 223 (1977); Szybalska & Szybalski, Proc. Natl. Acad. Sci. USA, 48: 2026 (1962); Lowy et al., Cell, 22: 817 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, CA) and pB-CMV from Stratagene (La Jolla, CA) represent non-limiting examples of an episomal vector that uses T-antigen and the SV40 origin of replication in lieu of EBNAl and oriP.

In some embodiments, the vector is an “integrating expression vector,” which may randomly integrate into the host cell's DNA or may include a recombination site to enable recombination between the expression vector and a specific site in the host cell's chromosomal DNA. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, CA) (e.g., pcDNA™5/FRT), or the cre-lox system, such as can be found in the pExchange-6 Core Vectors from Stratagene (La Jolla, CA). Examples of vectors that randomly integrate into host cell chromosomes include, for example, pcDNA3.1 (when introduced in the absence of T-antigen) from Invitrogen (Carlsbad, CA), and pCI or pFNI OA (ACT) FLEXI™ from Promega (Madison, WI).

In some embodiments, the vector is a viral vector. Representative viral expression vectors include, but are not limited to, the adenovirus-based vectors (e.g., the adenovirus-based Per.C6 system available from Crucell, Inc. (Leiden, The Netherlands)), lentivirus-based vectors (e.g., the lentiviral-based pLPl from Life Technologies (Carlsbad, CA)), and retroviral vectors (e.g., the pFB-ERV plus pCFB-EGSH from Stratagene (La Jolla, CA)). In a preferred embodiment, the viral vector is a lentivirus vector.

The vector comprising the inventive nucleic acid encoding the CAR can be introduced into a host cell that is capable of expressing the CAR encoded thereby, including any suitable prokaryotic or eukaryotic cell. Preferred host cells are those that can be easily and reliably grown, have reasonably fast growth rates, have well characterized expression systems, and can be transformed or transfected easily and efficiently.

As used herein, the term “host cell” refers to any type of cell that can contain the expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK 293 cells, and the like. In a preferred embodiment, the host cells are HEK 293 cells. In some embodiments, the HEK 293 cells are derived from the ATCC SD-3515 line. In some embodiments, the HEK 293 cells are derived from, the IU-VPF MCB line. In some embodiments, the HEK 293 cells are derived from the IU-VPF MWCB line. In some embodiments, the host cell can be a peripheral blood lymphocyte (PBL), a peripheral blood mononuclear cell (PBMC), or a natural killer (NK). Preferably, the host cell is a natural killer (NK) cell. More preferably, the host cell is a T-cell.

For purposes of amplifying or replicating the recombinant expression vector, the host cell may be a prokaryotic cell, e.g., a DH5a cell. For purposes of producing a virus from a viral expression vector, the host cell may be a eukaryotic cell, e.g., a HEK 293 cell. For purposes of producing a recombinant CAR, the host cell can be a mammalian cell. The host cell preferably is a human cell. The host cell can be of any cell type, can originate from any type of tissue, and can be of any developmental stage. Methods for selecting suitable mammalian host cells and methods for transformation, culture, amplification, screening, and purification of cells are known in the art.

In some embodiments, the disclosure provides an isolated host cell which expresses the nucleic acid sequence encoding the CARs described herein.

In some embodiments, the host cell is a T-cell. The T-cell of the disclosure can be any T-cell, such as a cultured T-cell, e.g., a primary T-cell, or a T-cell from a cultured T-cell line, or a T-cell obtained from a mammal. If obtained from a mammal, the T-cell can be obtained from numerous sources, including but not limited to blood, bone marrow, lymph node, the thymus, or other tissues or fluids. T-cells can also be enriched for or purified. The T-cell preferably is a human T-cell (e.g., isolated from a human). The T-cell can be of any developmental stage, including but not limited to, a CD4+/CD8+ double positive T-cell, a CD4+ helper T-cell, e.g., Th, and Th2 cells, a CD8 + T-cell (e.g., a cytotoxic T-cell), a tumor infiltrating cell, a memory T-cell, a naive T-cell, and the like. In one embodiment, the T-cell is a CD8+ T-cell or a CD4+ T-cell. T-cell lines are available from, e.g., the American Type Culture Collection (ATCC, Manassas, VA), and the German Collection of Microorganisms and Cell Cultures (DSMZ) and include, for example, Jurkat cells (ATCC TIB-152), Sup-Tl cells (ATCC CRL-1942), RPMI 8402 cells (DSMZ ACC-290), Karpas 45 cells (DSMZ ACC-545), and derivatives thereof.

In some embodiments, the host cell is a natural killer (NK) cell. NK cells are a type of cytotoxic lymphocyte that plays a role in the innate immune system. NK cells are defined as large granular lymphocytes and constitute a third kind of cells differentiated from the common lymphoid progenitor which also gives rise to B and T lymphocytes (see, e.g., Immunobiology, 5th ed., Janeway et al., eds., Garland Publishing, New York, NY (2001)). NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human). NK cell lines are available from, e.g., the American Type Culture Collection (ATCC, Manassas, VA) and include, for example, NK-92 cells (ATCC CRL-2407), NK92MI cells (ATCC CRL-2408), and derivatives thereof.

In some embodiments, the nucleic acid sequences encoding a CAR may be introduced into a cell by “transfection”, “transformation”, or “transduction”. “Transfection”, “transformation”, or “transduction”, as used herein, refer to the introduction of one or more exogenous polynucleotides into a host cell by using physical or chemical methods.

Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

### 5.6. Pharmaceutical Compositions and Formulations

Further provided by the present application are pharmaceutical compositions comprising any one of the anti-BCMA antibodies of the disclosure, or any one of the engineered immune effector cells comprising any one of the CARs (such as BCMA CARs) as described herein, and a pharmaceutically acceptable carrier. Pharmaceutical compositions can be prepared by mixing any of the immune effector cells described herein, having the desired degree of purity, with optional pharmaceutically acceptable carriers, excipients or stabilizers (Remington's Pharmaceutical Sciences 16th edition, Osol, A. Ed. (1980)), in the form of lyophilized formulations or aqueous solutions. In certain embodiments, a pharmaceutical composition of CAR-T cells further comprises an excipient selected from dimethylsulfoxide or dextran-40.

The compositions described herein may be administered as part of a pharmaceutical composition comprising one or more carriers. The choice of carrier will be determined in part by the particular nucleic acid sequence, vector, or host cells expressing the CAR disclosed herein, as well as by the particular method used to administer the nucleic acid sequence, vector, or host cells expressing the CAR disclosed herein. Accordingly, there are a variety of suitable formulations of the pharmaceutical composition of the disclosure.

For example, the pharmaceutical composition can contain preservatives. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition.

In addition, buffering agents may be used in the composition. Suitable buffering agents include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

The composition comprising the nucleic acid sequence encoding the CAR disclosed herein, or host cells expressing the CAR disclosed herein, can be formulated as an inclusion complex, such as cyclodextrin inclusion complex, or as a liposome. Liposomes can serve to target the host cells (e.g., T-cells or NK cells) or the nucleic acid sequence disclosed herein to a particular tissue. Liposomes also can be used to increase the half-life of the nucleic acid sequence disclosed herein. Many methods are available for preparing liposomes, such as those described in, for example, Szoka et al., Ann. Rev. Biophys. Bioeng., 9: 467 (1980), and U.S. Pat. Nos. 4,235,871; 4,501,728; 4,837,028; and 5,019,369. The composition can employ time-released, delayed release, and sustained release delivery systems such that the delivery of the composition disclosed herein occurs prior to, and with sufficient time to cause, sensitization of the site to be treated. Many types of release delivery systems are available and known to those of ordinary skill in the art. Such systems can avoid repeated administrations of the composition, thereby increasing convenience to the subject and the physician, and may be particularly suitable for certain composition embodiments of the disclosure.

In certain embodiments, the CAR-T cells are formulated at a dose of about 1.0×10⁵ to 2.0×10⁵ cells/kg, 1.5×10⁵ to 2.5×10⁵ cells/kg, 2.0×10⁵ to 3.0×10⁵ cells/kg, 2.5×10⁵ to 3.5×10⁵ cells/kg, 3.0×10⁵ to 4.0×10⁵ cells/kg, 3.5×10⁵ to 4.5×10⁵ cells/kg, 4.0×10⁵ to 5.0×10⁵ cells/kg, 4.5×10⁵ to 5.5×10⁵ cells/kg, 5.0×10⁵ to 6.0×10⁵ cells/kg, 5.5×10⁵ to 6.5×10⁵ cells/kg, 6.0×10⁵ to 7.0×10⁵ cells/kg, 6.5×10⁵ to 7.5×10⁵ cells/kg, 7.0×10⁵ to 8.0×10⁵ cells/kg, 7.5×10⁵ to 8.5×10⁵ cells/kg, 8.0×10⁵ to 9.0×10⁵ cells/kg, 8.5×10⁵ to 9.5×10⁵ cells/kg, or 9.0×10⁵ to 1.0×10⁶ cells/kg. In a preferred embodiment, the dose is formulated at approximately 0.75×10⁶ cells/kg. In certain embodiments, the CAR-T cells are formulated at a dose of less than 1.0×10⁸ cells per subject.

### 5.7. Methods of Treating Subjects

The present application further relates to methods and compositions for use in cell immunotherapy. In some embodiments, the cell immunotherapy is for treating cancer in a subject, including but not limited to hematological malignancies and solid tumors. In some embodiments, the subject is human. In some embodiments, the methods are suitable for treatment of adults and pediatric population, including all subsets of age, and can be used as any line of treatment, including first line or subsequent lines.

The engineered immune effector cells (such as CAR-T cells) described herein may be used in the method of treating cancer. In some embodiments, the immune effector cells are autologous. In some embodiments, the immune effector cells are allogeneic.

In certain embodiments, the CAR-T cells are administered at a dose of about 1.0×10⁵ to 2.0×10⁵ cells/kg, 1.5×10⁵ to 2.5×10⁵ cells/kg, 2.0×10⁵ to 3.0×10⁵ cells/kg, 2.5×10⁵ to 3.5×10⁵ cells/kg, 3.0×10⁵ to 4.0×10⁵ cells/kg, 3.5×10⁵ to 4.5×10⁵ cells/kg, 4.0×10⁵ to 5.0×10⁵ cells/kg, 4.5×10⁵ to 5.5×10⁵ cells/kg, 5.0×10⁵ to 6.0×10⁵ cells/kg, 5.5×10⁵ to 6.5×10⁵ cells/kg, 6.0×10⁵ to 7.0×10⁵ cells/kg, 6.5×10⁵ to 7.5×10⁵ cells/kg, 7.0×10⁵ to 8.0×10⁵ cells/kg, 7.5×10⁵ to 8.5×10⁵ cells/kg, 8.0×10⁵ to 9.0×10⁵ cells/kg, 8.5×10⁵ to 9.5×10⁵ cells/kg, 9.0×10⁵ to 1.0×10⁶ cells/kg, 1.0×10⁶ to 2.0×10⁶ cells/kg, 1.5×10⁶ to 2.5×10⁶ cells/kg, 2.0×10⁶ to 3.0×10⁶ cells/kg, 2.5×10⁶ to 3.5×10⁶ cells/kg, 3.0×10⁶ to 4.0×10⁶ cells/kg, 3.5×10⁶ to 4.5×10⁶ cells/kg, 4.0×10⁶ to 5.0×10⁶ cells/kg, 4.5×10⁶ to 5.5×10⁶ cells/kg, or 5.0×10⁶ to 6.0×10⁶ cells/kg. In a preferred embodiment, the dose comprises approximately 0.75×10⁶ cells/kg. In certain embodiments, the CAR-T cells are administered at a dose of about 1.0×10⁸ cells per subject.

In certain embodiments, the CAR-T cells are administered at a dose of less than 1.0×10⁸ cells per subject. In certain embodiments, the CAR-T cells are administered at a dose of about 3.0 to 4.0×10⁷ cells. In certain embodiments, the CAR-T cells are administered at a dose of about 3.5 to 4.5×10⁷ cells. In certain embodiments, the CAR-T cells are administered at a dose of about 4.0 to 5.0×10⁷ cells. In certain embodiments, the CAR-T cells are administered at a dose of about 4.5 to 5.5×10⁷ cells. In certain embodiments, the CAR-T cells are administered at a dose of about 5.0 to 6.0×10⁷ cells. In certain embodiments, the CAR-T cells are administered at a dose of about 5.5 to 6.5×10⁷ cells. In certain embodiments, the CAR-T cells are administered at a dose of about 6.0 to 7.0×10⁷ cells. In certain embodiments, the CAR-T cells are administered at a dose of about 6.5 to 7.5×10⁷ cells. In certain embodiments, the CAR-T cells are administered at a dose of about 7.0 to 8.0×10⁷ cells. In certain embodiments, the CAR-T cells are administered at a dose of about 7.5 to 8.5×10⁷ cells. In certain embodiments, the CAR-T cells are administered at a dose of about 8.0 to 9.0×10⁷ cells. In certain embodiments, the CAR-T cells are administered at a dose of about 8.5 to 9.5×10⁷ cells. In certain embodiments, the CAR-T cells are administered at a dose of about 9.0×10⁷ to 1.0×10′ cells.

In certain embodiments, the CAR-T cells are administered at a dose of about 0.693×10⁶ CAR-positive viable T-cells/kg. In certain embodiments, the CAR-T cells are administered at a dose of about 0.52×10⁶ CAR-positive viable T-cells/kg. In certain embodiments, the CAR-T cells are administered at a dose of about 0.94×10⁶ CAR-positive viable T-cells/kg. In certain embodiments, the CAR-T cells are administered at a dose of about 0.709×10⁶ CAR-positive viable T-cells/kg. In certain embodiments, the CAR-T cells are administered at a dose of about 0.51×10⁶ CAR-positive viable T-cells/kg. In certain embodiments, the CAR-T cells are administered at a dose of about 0.95×10⁶ CAR-positive viable T-cells/kg. In certain embodiments, the CAR-T cells are administered in an outpatient setting.

In certain embodiments, the CAR-T cells (e.g., at any of the foregoing doses) are administered in one or more intravenous infusions. In certain embodiments, said administration of said CAR-T cells is via a single intravenous infusion. In certain embodiments, said single intravenous infusion is administered using a single bag of said CAR-T cells. In certain embodiments, said administration of said single bag of said CAR-T cells is completed between the time at which said single bag of CAR-T cells is thawed and three hours after said single bag of CAR-T cells is thawed. In certain embodiments, single intravenous administration is administered using two bags of said CAR-T cells. In certain embodiments, said administration of each of said two bags of said CAR-T cells is completed between the time at which a first bag of said two bags of CAR-T cells is thawed and three hours after said first bag of CAR-T cells is thawed.

In certain embodiments, the time since the initial apheresis to the administration of CAR-T cells is less than 41, 47, 54, 61, 68, 75, 82, 89, 96, 103, 110, 117, 124, 131, 138, 145, 152, 159, 166 or 167 days. In certain embodiments, the time since the initial apheresis to the administration of CAR-T cells is greater than 41, 47, 54, 61, 68, 75, 82, 89, 96, 103, 110, 117, 124, 131, 138, 145, 152, 159, 166 or 167 days.

The composition comprising the host cells expressing the CAR-encoding nucleic acid sequence disclosed herein, or a vector comprising the CAR-encoding nucleic acid sequence disclosed herein, can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral”, as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection. Most preferably, the composition is administered by intravenous infusion.

The composition comprising the host cells expressing the CAR-encoding nucleic acid sequence disclosed herein, or a vector comprising the CAR-encoding nucleic acid sequence disclosed herein, can be administered with one or more additional therapeutic agents, which can be coadministered to the mammal. By “coadministering” is meant administering one or more additional therapeutic agents and the composition comprising the host cells disclosed herein or the vector disclosed herein sufficiently close in time such that the CAR disclosed herein can enhance the effect of one or more additional therapeutic agents, or vice versa. In this regard, the composition comprising the host cells disclosed herein or the vector disclosed herein can be administered first, and the one or more additional therapeutic agents can be administered second, or vice versa.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

In certain embodiments, a lymphodepleting regimen precedes said administration of CAR-T cells by approximately 5 days to approximately 7 days. In certain embodiments, lymphodepleting regimen is administered intravenously. In certain embodiments, said lymphodepleting regimen comprises administration of cyclophosphamide or administration of fludarabine. In certain embodiments, said cyclophosphamide is administered intravenously at 300 mg/m². In certain embodiments, said fludarabine is administered intravenously at 30 mg/m². In certain embodiments, a lymphodepleting regimen comprising cyclophosphamide administered intravenously at 300 mg/m² and fludarabine administered intravenously at 30 mg/m² precedes said administration of CAR-T cells by approximately 5 days to approximately 7 days.

In certain embodiments, the subject further receives bridging therapy, wherein said bridging therapy comprises short-term treatment with at least one bridging medicament between apheresis and said lymphodepleting regimen, and wherein said at least one bridging medicament had previously obtained an outcome of stable disease, minimal response, partial response, very good partial response, complete response or stringent complete response for the subject. In certain embodiments, the subject had an increase in tumor burden despite said bridging therapy. In certain embodiments, the subject had an increase in tumor burden of approximately 25% or greater despite said bridging therapy.

In certain embodiments, the subject is treated with pre-administration medication comprising an antipyretic and an antihistamine up to approximately 1 hour before said administration of said CAR-T cells. In certain embodiments, said antipyretic comprises either paracetamol or acetaminophen. In certain embodiments, said antipyretic is administered to the subject either orally or intravenously. In certain embodiments, said antipyretic is administered to the subject at a dosage of between 650 mg and 1000 mg. In certain embodiments, said antihistamine comprises diphenhydramine. In certain embodiments, said antihistamine is administered to the subject either orally or intravenously. In certain embodiments, said antihistamine is administered at a dosage of between 25 mg and 50 mg, or its equivalent. In certain embodiments, said antipyretic comprises either paracetamol or acetaminophen and said antipyretic is administered to the subject either orally or intravenously at a dosage of between 650 mg and 1000 mg, and wherein said antihistamine comprises diphenhydramine and said antihistamine is administered to the subject either orally or intravenously at a dosage of between 25 mg and 50 mg, or its equivalent.

In some embodiments, the method further comprises diagnosing said subject for cytokine release syndrome (CRS). In preferred embodiments, the diagnosis is made according to the American Society of Transplantation and Cellular Therapy (ASTCT), formerly the American Society for Blood and Marrow Transplantation (ASBMT) consensus grading. A non-limiting summary of the ASTCT consensus grading for CRS diagnosis is provided in Table 7. In some embodiments, the CRS is assessed by evaluating the levels of one or more of, or all of, IL-6, IL-10, IFN-γ, C-reactive protein (CRP) and ferritin.

In some embodiments, the method further comprises treating said subject for cytokine release syndrome (CRS). In some embodiments, the treatment of CRS is with an antipyretic. In some examples, the treatment of CRS is with anticytokine therapy. In some embodiments, the treatment of CRS occurs more than approximately 3 days following the infusion. In some embodiments, the treatment of CRS occurs without significantly reducing CAR-T cell expansion in vivo. In certain embodiments, said method further comprises treating said subject for cytokine release syndrome more than approximately 3 days following said administration of said CAR-T cells without significantly reducing expansion of said CAR-T cells in vivo. In some embodiments, the treatment of CRS comprises administering to the subject an IL-6R inhibitor. In some embodiments, the IL-6R inhibitor is an antibody. In some embodiments, the antibody inhibits IL-6R by binding its extracellular domain. In some embodiments, the IL-6R inhibitor prevents the binding of IL-6 to IL-6R. In some embodiments, the IL-6R inhibitor is tocilizumab. In some embodiments, the anticytokine therapy comprises administration of tocilizumab. In some embodiments, the anticytokine therapy comprises administration of steroids. In some embodiments, treatment for CRS comprises treatment with monoclonal antibodies other than tocilizumab. In some embodiments, the antibodies other than tocilizumab target cytokines. In some embodiments, the cytokine that the antibodies other than tocilizumab target is IL-1. In some embodiments, the IL-1 targeting antibody is Anakinra. In some embodiments, the cytokine that the antibodies other than tocilizumab target is TNFα. In some embodiments, the treatment of CRS comprises administering to the subject a corticosteroid. In some embodiments, the treatment of CRS comprises using a vasopressor. In some embodiments, the treatment of CRS comprises intubation or mechanical ventilation. In some embodiments, the treatment of CRS comprises administering to the subject cyclophosphamide. In some embodiments, the treatment of CRS comprises administering to the subject etanercept. In some embodiments, the treatment of CRS comprises administering to the subject levetiracetam. In some embodiments, the treatment of CRS comprises supportive care.

In some embodiments, the method further comprises diagnosing said subject for immune cell effector-associated neurotoxicity (ICANS). In some embodiments, the diagnosis is made according to the National Cancer Institute Common Terminology Criteria for Adverse Events (NCI CTCAE) criteria. In some embodiments, the diagnosis is made according to the NCI CTCAE criteria, Version 5.0. In some embodiments, the diagnosis is made according to the American Society of Transplantation and Cellular Therapy (ASTCT) consensus grading system. In some embodiments, the embodiments, there is neurotoxicity consistent with ICAN. A non-limiting summary of the ASTCT consensus grading system for ICANS diagnosis is provided in Table 8. In some embodiments, the treatment of ICANS comprises administering to the subject an IL-6R inhibitor. In some embodiments, the IL-6R inhibitor is an antibody. In some embodiments, the antibody inhibits IL-6R by binding its extracellular domain. In some embodiments, the IL-6R inhibitor prevents the binding of IL-6 to IL-6R. In some embodiments, the IL-6R inhibitor is tocilizumab. In some embodiments, the treatment of ICANS comprises administering to the subject an IL-1 inhibitor. In some embodiments the IL-1 inhibitor is an antibody. In a preferred embodiment, the IL-1 inhibiting antibody is Anakinra. In some embodiments, the treatment of ICANS comprises administering to the subject a corticosteroid. In some embodiments, the treatment of ICANS comprises administering to the subject levetiracetam. In some embodiments, the treatment of ICANS comprises administering to the subject dexamethasone. In some embodiments, the treatment of ICANS comprises administering to the subject methylprednisone sodium succinate. In some embodiments, the treatment of ICANS comprises administering to the subject pethidine. In some embodiments, the treatment of ICANS comprises administering to the subject one or more of, or all of, tocilizumab, Anakinra, a corticosteroid, levetiracetam, dexamethasone, methylprednisone sodium succinate or pethidine.

In some embodiments, the method further comprises diagnosing said subject for cytopenias. In some embodiments, the cytopenias comprise one or more of, or all of, lymphopenia, neutropenia, and thrombocytopenia. Without being bound by theory, a Grade 3 or Grade 4 but not a Grade 2 or lower lymphopenia is characterized by to a lymphocyte count less than 0.5×10⁹ cells per liter of a subject's blood sample, a Grade 3 or Grade 4 but not a Grade 2 or lower neutropenia is characterized by a neutrophil count less than 1000 cells per microliter of a subject's blood sample, and a Grade 3 or Grade 4 but not a Grade 2 or lower thrombocytopenia is characterized by a platelet count less than 50,000 cells per microliter of a subject's blood sample. In some embodiments, greater than 75% subjects with Grade 3 or Grade 4 lymphopenia following CAR-T cell administration recover to Grade 2 or lower lymphopenia 60 days following CAR-T cell administration. In some embodiments, greater than 80% subjects with Grade 3 or Grade 4 lymphopenia following CAR-T cell administration recover to Grade 2 or lower lymphopenia 60 days following CAR-T cell administration. In some embodiments, greater than 85% subjects with Grade 3 or Grade 4 lymphopenia following CAR-T cell administration recover to Grade 2 or lower lymphopenia 60 days following CAR-T cell administration. In some embodiments, greater than 90% subjects with Grade 3 or Grade 4 lymphopenia following CAR-T cell administration recover to Grade 2 or lower lymphopenia 60 days following CAR-T cell administration. In some embodiments, greater than 70% subjects with Grade 3 or Grade 4 neutropenia following CAR-T cell administration recover to Grade 2 or lower neutropenia 60 days following CAR-T cell administration. In some embodiments, greater than 75% subjects with Grade 3 or Grade 4 neutropenia following CAR-T cell administration recover to Grade 2 or lower neutropenia 60 days following CAR-T cell administration. In some embodiments, greater than 80% subjects with Grade 3 or Grade 4 neutropenia following CAR-T cell administration recover to Grade 2 or lower neutropenia 60 days following CAR-T cell administration. In some embodiments, greater than 85% subjects with Grade 3 or Grade 4 neutropenia following CAR-T cell administration recover to Grade 2 or lower neutropenia 60 days following CAR-T cell administration. In some embodiments, greater than 30% subjects with Grade 3 or Grade 4 thrombocytopenia following CAR-T cell administration recover to Grade 2 or lower thrombocytopenia 60 days following CAR-T cell administration. In some embodiments, greater than 34% subjects with Grade 3 or Grade 4 thrombocytopenia following CAR-T cell administration recover to Grade 2 or lower thrombocytopenia 60 days following CAR-T cell administration. In some embodiments, greater than 38% subjects with Grade 3 or Grade 4 thrombocytopenia following CAR-T cell administration recover to Grade 2 or lower thrombocytopenia 60 days following CAR-T cell administration. In some embodiments, greater than 42% subjects with Grade 3 or Grade 4 thrombocytopenia following CAR-T cell administration recover to Grade 2 or lower thrombocytopenia 60 days following CAR-T cell administration.

Once the composition comprising host cells expressing the CAR-encoding nucleic acid sequence disclosed herein, or a vector comprising the CAR-encoding nucleic acid sequence disclosed herein, is administered to a mammal (e.g., a human), the biological activity of the CAR can be measured by any suitable method known in the art. In accordance with the method disclosed herein, the CAR binds to BCMA on the multiple myeloma cells, and the multiple myeloma cells are destroyed. Binding of the CAR to BCMA on the surface of multiple myeloma cells can be assayed using any suitable method known in the art, including, for example, ELISA and flow cytometry. The ability of the CAR to destroy multiple myeloma cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). The biological activity of the CAR also can be measured by assaying expression of certain cytokines, such as CD 107a, IFNγ, IL-2, and TNF.

The methods described herein may be used for treating various cancers, including both solid cancer and liquid cancer. In certain embodiments, the methods are used to treat multiple myeloma. The methods described herein may be used as a first therapy, second therapy, third therapy, or combination therapy with other types of cancer therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

In certain embodiments, the cancer is multiple myeloma. In certain embodiments, the cancer is stage I, stage II or stage III, and/or stage A or stage B multiple myeloma based on the Durie-Salmon staging system. In certain embodiments, the cancer is stage I, stage II or stage III multiple myeloma based on the International staging system published by the International Myeloma Working Group (IMWG). In some embodiments, the multiple myeloma is progressive.

In certain embodiments, the subject received prior treatment with at least one prior line of therapy. In certain embodiments, the at least one prior line of therapy comprises treatment with a medicament that is a proteasomal inhibitor (PI). Non-limiting examples of a PI include bortezomib, carfilzomib and ixazomib. In certain embodiments, the at least one prior line of therapy comprises treatment with a medicament that is an immunomodulatory drug (IMiD). Non-limiting examples of an IMiD include lenalidomide, pomalidomide and thalidomide. In certain embodiments, the at least one prior line of therapy comprises treatment with a medicament that is a corticosteroid. Non-limiting examples of a corticosteroid include dexamethasone and prednisone. In certain embodiments, at least one prior line of therapy comprises treatment with a medicament that is an alkylating agent. In certain embodiments, at least one prior line of therapy comprises treatment with a medicament that is an anthracycline. In certain embodiments at least one prior line of therapy comprises treatment with a medicament that is an anti-CD38 antibody. Non-limiting examples of an anti-CD38 antibody include daratumumab, isatuximab and the investigational antibody TAK-079. In certain embodiments, at least one prior line of therapy comprises treatment with a medicament that is elotuzumab. In certain embodiments, at least one prior line of therapy comprises treatment with a medicament that is panobinostat. In certain embodiments, the subject has relapsed after said at least one prior line of therapy. In certain embodiments, the cancer is refractory to one or more of, or all of, bortezomib, carfilzomib, ixazomib, lenalidomide, pomalidomide, thalidomide, dexamethasone, prednisone, alkylating agents, daratumumab, isatuximab, TAK-079, elotuzumab and panobinostat. In certain embodiments prior lines of therapy include surgery, radiotherapy, or autologous or allogeneic transplant, or any combination of such treatments.

In some embodiments, the multiple myeloma is refractory to at least two medicaments. In some embodiments, the multiple myeloma is refractory to at least three medicaments. In some embodiments, the multiple myeloma is refractory to at least four medicaments. In some embodiments, the multiple myeloma is refractory to at least five medicaments.

In some embodiments, the subject has bone marrow plasma cells of between approximately 10% and approximately 30% before said administration of said CAR-T cells.

Methods of Treating Lenalidomide-Refractory Subjects

In one aspect is provided a method of treating a subject, said method comprising administering to the subject a composition comprising a therapeutically effective number of T cells comprising a chimeric antigen receptor (CAR), wherein said subject has multiple myeloma and is lenalidomide-refractory. In some embodiments, the subject has received prior treatment with one, two or three prior lines of therapy.

In some embodiments, the multiple myeloma is refractory to the last line of therapy. In some embodiments, the subject has relapsed after said one, two or three prior lines of therapy. In some embodiments, the subject received prior treatment with at least one prior line of therapy comprising treatment with lenalidomide and at least one non-lenalidomide medicament, said at least one non-lenalidomide medicament comprising at least one of a proteasomal inhibitor, an immunomodulatory drug or an anti-CD38 antibody. In some embodiments, the subject has had no prior exposure to a BCMA-targeting medicament. In some embodiments, the subject received prior treatment with at least two prior lines of therapy. In some embodiments, the subject received prior treatment with three prior lines of therapy.

In some embodiments, the subject received prior treatment with dexamethasone, an alkylating agent or daratumumab. In some embodiments, the multiple myeloma is refractory to three classes of medicaments.

Methods of Treating Subjects with Prior Early Relapse

In one aspect is provided a method of treating a subject, said method comprising administering to the subject a composition comprising a therapeutically effective number of T cells comprising a chimeric antigen receptor (CAR), wherein said subject has multiple myeloma and has had a prior early relapse. The term “prior early relapse” means disease progression per International Myeloma Working Group (IMWG)-based response criteria either: (i) between the time of treatment with autologous stem cell transplantation (ASCT) and approximately 12 months after said treatment with autologous stem cell transplantation (ASCT), for participants who have had autologous stem cell transplantation (ASCT); or (ii) between the time of start of anti-myeloma therapy and approximately 12 months from the start of anti-myeloma therapy, for participants who have not had autologous stem cell transplantation (ASCT).

In some embodiments, the subject has received prior treatment with one prior line of therapy. In some embodiments, said one prior line of therapy comprising treatment with at least two medicaments. In some embodiments, said at least two medicaments comprise a proteasomal inhibitor and an immunomodulatory drug. In some embodiments, the subject was additionally treated with an anti-CD38 antibody. In some embodiments, the subject has had no prior exposure to a BCMA-targeting medicament. In some embodiments, the multiple myeloma is refractory to at least one medicament.

Methods of Treating Subjects with Prior Non-Cellular BCMA-Targeted Treatment

In one aspect is provided a method of treating a subject, said method comprising administering to the subject a composition comprising a therapeutically effective number of T cells comprising a chimeric antigen receptor (CAR), wherein said subject has multiple myeloma and has received at least one prior line of therapy comprising treatment with a non-cellular BCMA-targeting medicament. In some embodiments, said at least one prior line of therapy comprises treatment with at least four medicaments, wherein said at least four medicaments comprises a non-cellular BCMA-targeting medicament. In some embodiments, said at least four medicaments further comprises a proteasomal inhibitor, an immunomodulatory drug and an anti-CD38 antibody.

In some embodiments, the subject received prior treatment with at least two prior lines of therapy, at least three prior lines of therapy, at least four prior lines of therapy, at least five prior lines of therapy, at least six prior lines of therapy, at least seven prior lines of therapy, at least eight prior lines of therapy, at least nine prior lines of therapy, at least ten prior lines of therapy, at least eleven prior lines of therapy, or at least twelve prior lines of therapy. In some embodiments, the subject has relapsed after said at least one prior line of therapy, at least two prior lines of therapy, at least three prior lines of therapy, at least four prior lines of therapy, at least five prior lines of therapy, at least six prior lines of therapy, at least seven prior lines of therapy, at least eight prior lines of therapy, at least nine prior lines of therapy, at least ten prior lines of therapy, at least eleven prior lines of therapy, or at least twelve prior lines of therapy.

### 5.8. Kits and Articles of Manufacture

Any of the compositions described herein may be comprised in a kit. In some embodiments, engineered immortalized CAR-T cells are provided in the kit, which also may include reagents suitable for expanding the cells, such as media.

In a non-limiting example, a chimeric receptor expression construct, one or more reagents to generate a chimeric receptor expression construct, cells for transfection of the expression construct, and/or one or more instruments to obtain immortalized T cells for transfection of the expression construct (such an instrument may be a syringe, pipette, forceps, and/or any such medically approved apparatus).

In some aspects, the kit comprises reagents or apparatuses for electroporation of cells.

In some embodiments, the kit comprises artificial antigen presenting cells.

The kits may comprise one or more suitably aliquoted compositions of the present disclosure or reagents to generate compositions of the disclosure. The components of the kits may be packaged either in aqueous media or in lyophilized form. The container means of the kits may include at least one vial, test tube, flask, bottle, syringe, or other container means, into which a component may he placed, and preferably, suitably aliquoted. Where there is more than one component in the kit, the kit also will generally contain a second, third, or other additional container into which the additional components may be separately placed. However, various combinations of components may be comprised in a vial. The kits of the present disclosure also will typically include a means for containing the chimeric receptor construct and any other reagent containers in close confinement for commercial sale. Such containers may include injection or blow molded plastic containers into which the desired vials are retained, for example.

For the sake of conciseness, certain abbreviations are used herein. One example is the single letter abbreviation to represent amino acid residues. The amino acids and their corresponding three letter and single letter abbreviations are as follows:

| Amino acid | Three letter | One letter |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### 6. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 6.1. Example 1—Ciltacabtagene Autoleucel

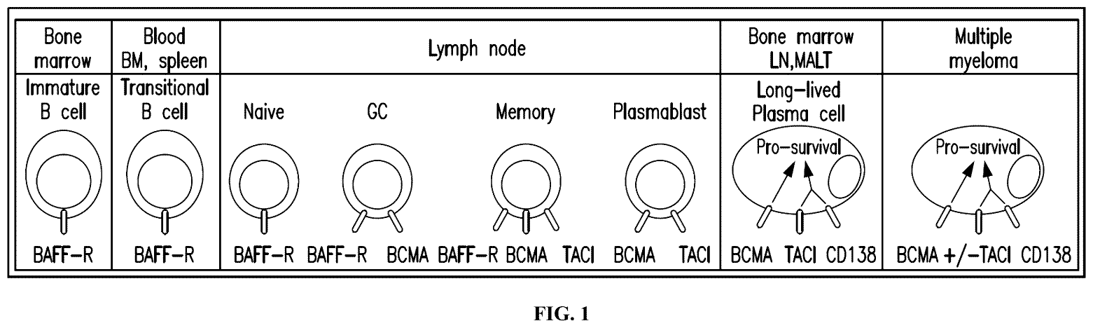

B cell maturation antigen (BCMA, also known as CD269 and TNFRSF17) is a 20 kilodalton, type III membrane protein that is part of the tumor necrosis receptor superfamily. BCMA is a cell surface antigen that is predominantly expressed in B-lineage cells at high levels. FIG. 1 shows the expression of BCMA on various immune-derived cells. Comparative studies have shown a lack of BCMA in most normal tissues and absence of expression on CD34-positive hematopoietic stem cells. BCMA binds 2 ligands that induce B cell proliferation, and plays a critical role in B cell maturation and subsequent differentiation into plasma cells. The selective expression and the biological importance for the proliferation and survival of myeloma cells makes BCMA a promising target for CAR-T based immunotherapy, ciltacabtagene autoleucel.

Ciltacabtagene autoleucel is an autologous chimeric antigen receptor T cell (CAR-T) therapy that targets BCMA. The ciltacabtagene autoleucel chimeric antigen receptor (CAR) comprises two B-cell maturation antigen (BCMA)-targeting VHH domains designed to confer avidity. A map of the construct is depicted in FIG. 2. Ciltacabtagene autoleucel includes a VHH domain comprising the amino acid sequence set forth in SEQ ID NO: 2 and a VHH domain comprising the amino acid sequence set forth in SEQ ID NO: 4.

### 6.2. Example 2. Method of Treatment with Ciltacabtagene Autoleucel (Ciltacel)

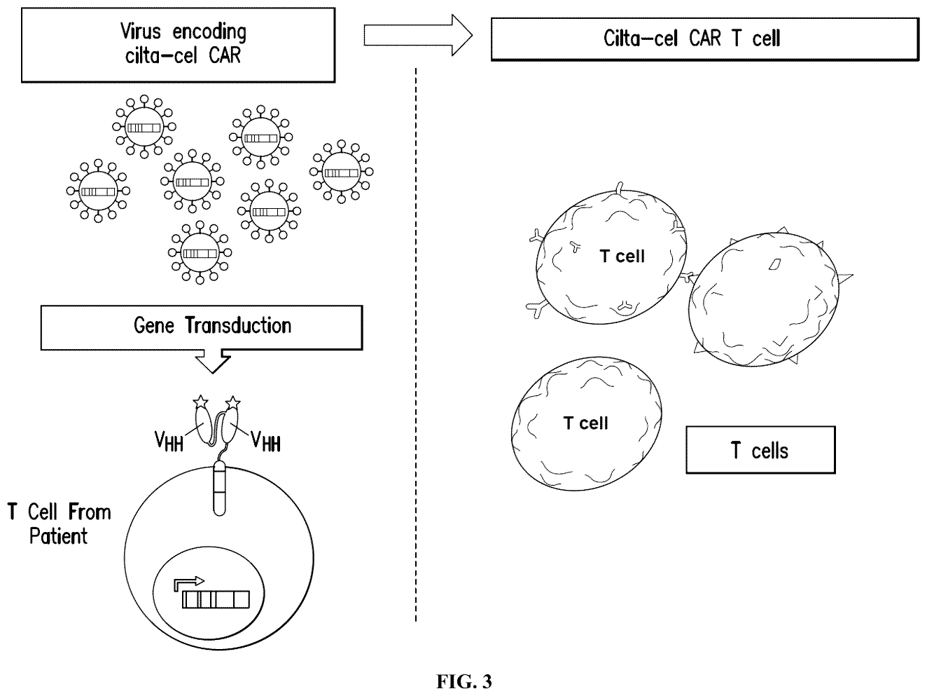

Herein, we describe a Phase 1b-2, open-label, multicenter study that we conducted to evaluate the safety and efficacy of ciltacabtagene autoleucel in adult subjects with relapsed or refractory multiple myeloma. In the Phase 1b portion of the study, a recommended Phase 2 dose (RP2D) of cilta-cel was confirmed. In Phase 2, subjects were treated at the RP2D established from Phase 1b. The objective of the phase 2 portion of the study was to further establish the safety and efficacy of cilta-cel. A schematic overview of the study flow chart, which consists of a lymphodepleting regimen prior to cilta-cel infusion, is depicted in FIG. 3.

The first analysis was conducted approximately 6 months after the last subject received their initial dose of cilta-cel. This report is generated from the protocol-specified first analysis. A summary of the subjects enrolled in the study is presented in Table 1, in which percentages were calculated with the number of subjects in the all enrolled analysis set as denominator. A total of 113 subjects (Phase 1b: 35; Phase 2: 78) were enrolled (apheresed) in the US, out of which 101 subjects (Phase 1b: 30; Phase 2: 71) received conditioning regimen and 97 subjects (Phase 1b: 29; Phase 2: 68) received cilta-cel infusion and received it at the targeted RP2D. These 97 subjects constituted the all treated analysis set, which is the basis for all efficacy and safety analyses presented below. At the clinical cutoff, the median duration of follow-up, based on Kaplan-Meier product limit estimate, for the all treated analysis set was 12.4 months. A summary of the study's duration of follow-up is presented in Table 2, which lists duration of follow up relative to the date of the initial cilta-cel infusion (Day 1).

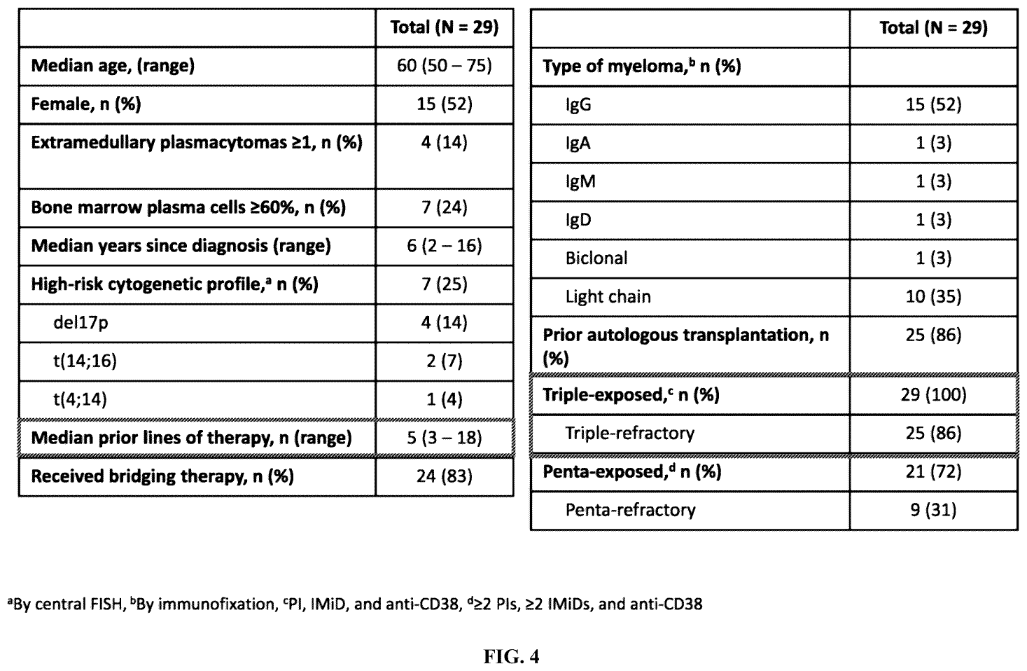

The patient population was screened to include those with relapsed or Refractory Multiple Myeloma, with 3 prior lines or double refractory to PI/IMiD and prior PI, IMiD, anti-CD38 exposure, where PI is a proteasomal inhibitor and IMiD is an immunomodulatory drug. Another possible medicament is an alkylating agent (ALKY). A summary of prior therapies received by the study subjects is presented in Table 3, and a summary of the refractory status of our study subjects to prior multiple myeloma therapies is presented in Table 4. Eligible patients were ≥18 years of age, had a diagnosis of MM per International Myeloma Working Group (IMWG) diagnostic criteria, measurable disease at baseline, and an Eastern Cooperative Oncology Group (ECOG) performance status score of 0, 1 or 2. Demographic and disease characteristics of the patient population in the Phase 1b portion of the study is shown in FIG. 4.

Eligible subjects underwent apheresis for collection of peripheral blood mononuclear cells (PBMC). Study enrollment was defined at the day of apheresis. The ciltacabtagene autoleucel drug product (DP) was generated from T cells selected from the apheresis. Subjects for whom apheresis or manufacturing failed were allowed a second attempt at apheresis.

Bridging therapy (anti-plasma cell directed treatment between apheresis and the first dose of the conditioning regimen) was allowed when clinically indicated (i.e., to maintain disease stability while waiting for manufacturing of ciltacabtagene autoleucel). Additional cycles of bridging therapy were considered based on the subject's clinical status and timing of availability of CAR− T product. A bridging therapy is defined as short-term treatment which had previously generated at least a response of stable disease for the subject.

After meeting safety criteria for treatment, subjects were administered a conditioning regimen to help achieve to lymphodepletion and promote CAR-T cell expansion in the subject. The lymphodepleting regimen comprised intravenous (IV) administration of cyclophosphamide 300 mg/m² and fludarabine 30 mg/m² daily for 3 days. Cyclophosphamide 300 mg/m² and fludarabine 30 mg/m² before cilta-cel infusion is consistent with the lymphodepletion regimen used in the marketed CAR-T products Kymriah and Yescarta.

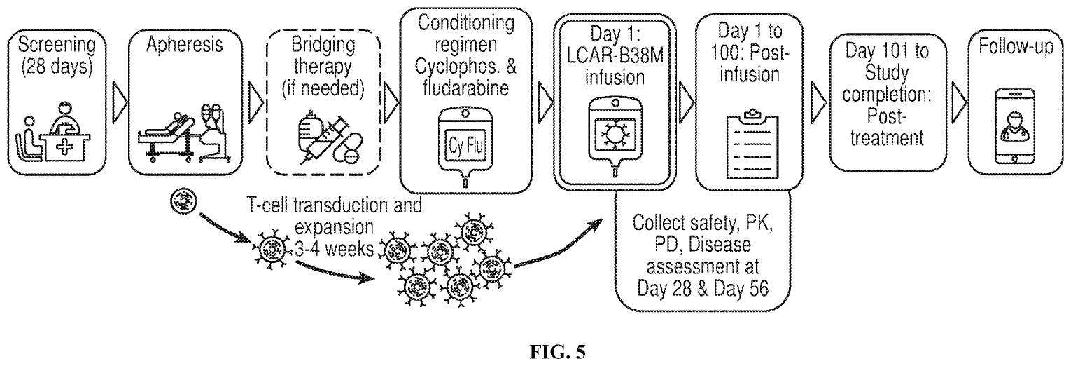

5 to 7 days after start of the conditioning regimen, cilta-cel, which had been prepared from apheresed material via viral transduction as shown in FIG. 5, was administered on a day defined as Day 1. Approximately one hour prior to cilta-cel infusion, subjects received premedication. Corticosteroids were not be used during pre-infusion. Pre-infusion medication is listed in Table 5. Following treatment with the pre-infusion medication, cilta-cel administration was performed in a single infusion at a total targeted dose of 0.75×10⁶ CAR-positive viable T cells/kg (range: 0.5-1.0×10⁶ CAR-positive viable T cells/kg) with a maximum total dose of 1.0×10⁸ CAR-positive viable T cells.

A dose of ciltacabtagene autoleucel was contained in either 1 or 2 cryopreserved patient-specific infusion bags. The timing of cilta-cel thaw was coordinated with the timing of the infusion. The infusion time was confirmed in advance, and the start time for thaw was adjusted so that cilta-cel was available for infusion when the patient would have been ready. If more than one bag was received for the treatment infusion, 1 bag was thawed at a time. The thawing/infusion of the next bag was made to wait until it was determined that the previous bag had been safely administered.

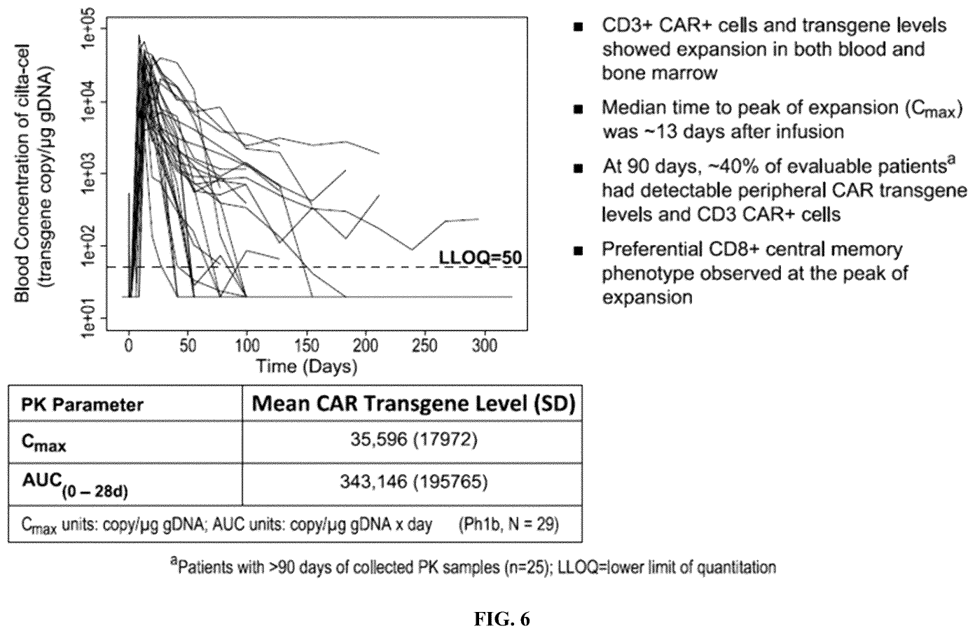

The post-infusion period started after the completion of cilta-cel infusion on Day 1 and lasted until Day 100. The post-treatment period started on Day 101 and lasted until study completion, defined as 2 years after the last subject had received his or her initial dose of cilta-cel. The expansion and persistence of cilta-cel as measured by blood concentration is summarized in FIG. 6.

### 6.3. Example 3. Evaluation of Efficacy of Method of Treatment with Ciltacabtagene Autoleucel

Using the IMWG-based response criteria summarized in Table 6, this study classified a response, in order from better to worse, as either a stringent complete response (sCR), a complete response (CR), a very good partial response (VGPR), a partial response (PR), a minimal response (MR), a stable disease or a progressive disease. Disease progression was consistently documented across clinical study sites. The tests performed to assess IMWG-based response criteria are as follows:

Myeloma Protein Measurements in Serum and Urine: Myeloma protein (M-protein) measurements were made using the following tests from blood and 24-hour urine samples: serum quantitative Ig, serum protein electrophoresis (SPEP), serum immunofixation electrophoresis, serum FLC assay (for subject in suspected CR/sCR and every disease assessment for subjects with serum FLC only disease), 24-hour urine M-protein quantitation by electrophoresis (UPEP), urine immunofixation electrophoresis, serum β 2-microglobulin. Disease progression based on one of the laboratory tests alone were confirmed by at least 1 repeat investigation. Disease evaluations continued beyond relapse from CR until disease progression was confirmed. Serum and urine immunofixation and serum free light chain (FLC) assays were performed at screening and thereafter when a CR was suspected (when serum or 24-hour urine M-protein electrophoresis [by SPEP or UPEP] were 0 or non-quantifiable). For subjects with light chain multiple myeloma, serum and urine immunofixation tests were performed routinely.

Serum Calcium Corrected for Albumin: Blood samples for calculating serum calcium corrected for albumin were collected and analyzed until the development of confirmed disease progression; development of hypercalcemia (corrected serum calcium>11.5 mg/dL [>2.9 mmol/L]) may indicate disease progression or relapse if it is not attributable to any other cause. Calcium binds to albumin and only the unbound (free) calcium is biologically active; therefore, the serum calcium level must be adjusted for abnormal albumin levels (“corrected serum calcium”).

Bone Marrow Examination: Bone marrow aspirate or biopsy was performed for clinical assessments. Bone marrow aspirate was performed for biomarker evaluations. Clinical staging (morphology, cytogenetics, and immunohistochemistry or immunofluorescence or flow cytometry) was done. A portion of the bone marrow aspirate was immunophenotyped and monitor for BCMA, checkpoint ligand expression in CD138-positive multiple myeloma cells, and checkpoint expression on T cells. If feasible, bone marrow aspirate also was performed to confirm CR and sCR and at disease progression. Additionally, since minimal residual disease (MRD) negativity was being evaluated as a potential surrogate for PFS and OS in multiple myeloma treatment, MRD was monitored in subjects using next generation sequencing (NGS) on bone marrow aspirate DNA Baseline bone marrow aspirates were used to define the myeloma clones, and post-treatment samples were used to evaluate MRD negativity. A fresh bone marrow aspirate was collected prior to the first dose of conditioning regimen (≤7 days).

Skeletal Survey: A skeletal survey (including skull, entire vertebral column, pelvis, chest, humeri, femora, and any other bones for which the investigator suspects involvement by disease) was performed during the screening phase and evaluated by either roentgenography (“X-rays”) or low-dose computed tomography (CT) scans without the use of IV contrast. If a CT scan was used, it was of diagnostic quality. Following cilta-cel infusion, and before disease progression was confirmed, X-rays or CT scans were performed locally, whenever clinically indicated based on symptoms, to document response or progression. Magnetic resonance imaging (MRI) was an acceptable method for evaluation of bone disease, and was included at discretion; however, it did not replace the skeletal survey. If a radionuclide bone scan was used at screening, in addition to the complete skeletal survey, then both methods were used to document disease status. These tests were performed at the same time. A radionuclide bone scan did not replace a complete skeletal survey. If a subject presented with disease progression manifested by symptoms of pain due to bone changes, then disease progression was documented by skeletal survey or other radiographs, depending on the symptoms that the subject experiences. If the diagnosis of disease progression was obvious by radiographic investigations, then no repeat confirmatory X-rays were thought necessary to perform. If changes were equivocal, then a repeat X-ray was performed in 1 to 3 weeks.

Documentation of Extramedullary Plasmacytomas: Sites of known extramedullary plasmacytomas were documented≤14 days prior to the first dose of the conditioning regimen. Clinical examination or MRI were used to document extramedullary sites of disease. CT scan evaluations were considered an acceptable alternative if there was no contraindication to the use of IV contrast. Positron emission tomography scan or ultrasound tests were not acceptable to document the size of extramedullary plasmacytomas. However, PET/CT fusion scans were optionally used to document extramedullary plasmacytomas if the CT component of the PET/CT fusion scan was of sufficient diagnostic quality. Extramedullary plasmacytomas were assessed for all subjects with a history of plasmacytomas or if clinically indicated at ≤14 days prior to the first dose of the conditioning regimen, by clinical examination or radiologic imaging. Assessment of measurable sites of extramedullary disease were performed, measured, and evaluated locally every 4 weeks (for physical examination) for subjects with a history of plasmacytomas or as clinically indicated during treatment for other subjects until development of confirmed CR or confirmed disease progression. If assessment could only be performed radiologically, then evaluation of extramedullary plasmacytomas was done every 12 weeks. Irradiated or excised lesions were considered not measurable and were monitored only for disease progression. To qualify for VGPR or PR/minimal response (MR), the sum of products of the perpendicular diameters of the existing extramedullary plasmacytomas must have decreased by over 90% or at least 50%, respectively, and new plasmacytomas must not have developed. To qualify for disease progression, either the sum of products of the perpendicular diameters of the existing extramedullary plasmacytomas must have increased by at least 50%, or the longest diameter of previous lesion>I cm in short axis must have increased at least 50%, or a new plasmacytoma must have developed. When not all existing extramedullary plasmacytomas were reported, but the sum of products of the perpendicular diameters of the reported plasmacytomas had increased by at least 50%, then the criterion for disease progression was met.

If it was determined that the study treatment interfered with the immunofixation assay, CR was defined as the disappearance of the original M-protein associated with multiple myeloma on immunofixation, and the determination of CR was not affected by unrelated M-proteins secondary to the study treatment.

Study endpoints, as assessed by an independent review committee (TRC), were as follows:

MRD was assessed at baseline, day 28, and 6-, 12-, 18-, and 24-month follow-ups using next-generation sequencing (clonoSEQ version 2.0) (Adaptive Biotechnologies, Seattle, WA, USA) in patients at the time of suspected complete response, and then every 12 months until disease progression for patients who remained on study. MRD negativity was assessed in samples that passed calibration or quality control and included sufficient cells for evaluation at the testing threshold of 10⁻⁵. Durability of MRD-negative status was evaluated by estimating MRD negativity rates at 6- and 12-month follow-ups.

Clinical benefit rate (CBR) was defined as the proportion of subjects who achieved a MR or better according to the IMWG criteria (sCR+CR+VGPR+PR+MR).

Overall response rate (ORR) was defined as the proportion of subjects who achieved a PR or better according to the IMWG criteria (sCR+CR+VGPR+PR).

VGPR or better response rate was defined as the proportion of subjects who achieve a VGPR or better response according to the IMWG criteria (sCR+CR+VGPR).

Duration of response (DOR) was calculated among responders (with a PR or better response) from the date of initial documentation of a response (PR or better) to the date of first documented evidence of progressive disease, as defined in the IMWG criteria. Relapse from CR by positive immunofixation or trace amount of M-protein was not considered as disease progression. Disease evaluations continued beyond relapse from CR until disease progression was confirmed.

Time to response (TTR) was defined as the time between date of the initial infusion of cilta-cel and the first efficacy evaluation at which the subject had met all criteria for PR or better.

Progression-free survival (PFS) was defined as the time from the date of the initial infusion of cilta-cel to the date of first documented disease progression, as defined in the IMWG criteria, or death due to any cause, whichever occurred first.

Overall survival (OS) was measured from the date of the initial infusion of cilta-cel to the date of the subject's death.

For ORR, the response rate and its 95% exact confidence interval (CI) was calculated based on binomial distribution, and the null hypothesis was rejected if the lower bound of the confidence interval exceeded 30%. Analysis of VGPR or better response rate, DOR, PFS, and OS was conducted at the same cutoff as the ORR. Time-to-event efficacy endpoints (DOR, PFS, and OS) were estimated using the Kaplan-Meier method. The distribution (median and Kaplan-Meier curves) of DOR was provided using Kaplan-Meier estimates. Similar analysis was performed for OS, PFS, and TTR.

### 6.4. Example 4. Evaluation of Safety of Method of Treatment with Ciltacabtagene Autoleucel

Adverse events were followed, reported and graded according to the National Cancer Institute Common Terminology Criteria for Adverse Events (NCI-CTCAE Version 5.0), with the exception of CRS and CAR-T cell-related neurotoxicity (e.g., ICANS). CRS was evaluated according to the ASTCT consensus grading, summarized in Table 7. At the first sign of CRS (such as fever), subjects were immediately hospitalized for evaluation. Tocilizumab intervention was discretionally used to treat subjects presenting symptoms of fever when other sources of fever had been eliminated. Tocilizumab was discretionally used for early treatment in subjects at high risk of severe CRS (for example, high baseline tumor burden, early fever onset, or persistent fever after 24 hours of symptomatic treatment). Other monoclonal antibodies targeting cytokines (for example, anti-ILI and/or anti-TNFa) were optionally used, especially for cases of CRS which did not respond to tocilizumab.

CAR-T cell-related neurotoxicity (e.g., ICANS) was graded using the ASTCT consensus grading, summarized in Table 8. Additionally, all individual symptoms of CRS (e.g., fever, hypotension) and ICANS (e.g., depressed level of consciousness, seizures) were captured as individual adverse events and graded by CTCAE criteria. Neurotoxicity that was not temporarily associated with CRS, or any other neurologic adverse events that did not qualify as ICANS, were graded by CTCAE criteria. Any adverse event or serious adverse event not listed in the NCI CTCAE Version 5.0 was graded according to investigator clinical judgment by using the standard grades as follows:

Grade 1: Mild; asymptomatic or mild symptoms; clinical or diagnostic observations only; intervention not indicated.

Grade 2: Moderate; minimal, local or noninvasive intervention indicated; limiting age-appropriate instrumental activities of daily living.

Grade 3: Severe or medically significant but not immediately life-threatening; hospitalization or prolongation of hospitalization indicated; disabling; limiting self-care activities of daily living.

Grade 4: Life-threatening consequences; urgent intervention indicated.

Grade 5: Death related to adverse event.

The response and duration of response of responders in the all treated analysis set at a median follow-up time of 12.4 months, based on Independent Review Committee (IRC) assessment, was analyzed. The overall best response for subjects in the all treated analysis set is summarized in Table 9. In the all treated analysis set, based on IRC assessment 94 subjects (96.9%) achieved a response of PR or better, 65 subjects (67.0%) achieved complete response (CR) or better, CBR was 96.9%. The deep and durable response induced by ciltacabtagene autoleucel were demonstrated by a VGPR or better rate of 92.8% and a CR or better rate of 67.0%, and a median DOR not reached with a median follow-up of 12.4 months at the time of clinical cutoff. The metrics used to evaluate ciltacabtagene autoleucel efficacy are summarized below:

Tumor burden reduction: Tumor burden was reduced in 100% of subjects.

Overall Response Rate (ORR): 96.9% of subjects had overall responses, with 95% exact CI (91.2%, 99.4%).

VGPR or better: 90 subjects (92.8% of subjects) achieved VGPR (very good partial response) or better.

Duration of Response (DOR): Median DOR was not reached with 95% CI (15.9, NE) months; the probabilities of the responders remaining in response at 9 months and 12 months were 80.2% (95% CI: 70.4%, 87.0%) and 68.2% (95% CI: 54.4%, 78.6%), respectively. DOR for all responders in the all treated analysis set is summarized in Table 10.

Time to Response (TTR): Median time to first response (PR or better) and median time to best response were 0.95 and 2.56 months, respectively.

Progression-Free Survival (PFS): Median PFS was not reached with 95% CI (16.79, NE) months; 9-month and 12-month PFS rates (95% CI) were 80.3% (70.9%, 87.0%) and 76.6% (66.0%, 84.3%), respectively. A summary of the PFS in the all treated analysis set is presented in Table 11.

Overall Survival (OS): Fourteen subjects (14.4%) had died at the time of clinical cutoff Nine-month and 12-month overall survival rates (95% CI) were 90.7% (82.8%, 95.0%) and 88.5% (80.2%, 93.5%), respectively. OS based on the all treated analysis set is summarized in Table 12.

Minimal Residual Disease (MRD) negative rate (at 10⁻⁵ sensitivity level): MRD negative rate was 54.6% (95% CI: 44.2%, 64.8%) and 33 (34.0%) subjects achieved MRD-negative CR/sCR. Summaries of overall MRD negativity rate at 10⁻⁵ in the bone marrow are presented, for all subjects in the all treated analysis set in Table 13 and for subjects with evaluable sample a 10⁻⁵ in the all treated analysis set in Table 14. Evaluable samples were those that passed calibration and quality control, and included sufficient cells for evaluation at the respective testing threshold.

Ciltacabtagene autoleucel was determined to have a safety profile consistent with the mechanism of action of CAR-T therapy.

CRS: CAR-T cell-related adverse events of CRS were common (94.8%) but most were low grade. All-grade CRS was reported for 92 (94.8%) subjects, as evaluated by the ASTCT consensus grading system. All events of CRS had recovered, with the exception of I (1.1%) fatal event from a subject with a 97-day duration of CRS. A summary of treatment-emergent CRS events in the all treated analysis set is presented in Table 15.

Immune Effector Cell-Associated Neurotoxicity (ICANS): All-grade ICANS was reported for 16 (16.5%) subjects, as evaluated by the ASTCT consensus grading system. All events had recovered. A summary of ICANS, with onset after cilta-cel infusion, in the all treated analysis set is presented in Table 16.

Cytopenias: Grade 3 or 4 cytopenias were common in the post-infusion period, including lymphopenia, neutropenia, thrombocytopenia, but the majority of these events recovered by Day 60. 96 (99.0%), 95 (97.9%) and 60 (61.9%) subjects had Grade 3 or 4 lymphopenia, neutropenia, and thrombocytopenia, respectively, in the first 100 days after cilta-cel infusion. 88 (90.7%), 85 (87.6%), and 41 (42.3%) subjects had their initial Grade 3 or 4 events recovered to Grade 2 or lower by Day 60 for lymphopenia, neutropenia, and thrombocytopenia, respectively. A summary of cytopenias following treatment with cilta-cel in the all treated analysis set is presented in Table 17.

In conclusion, single-agent and one-time infusion of ciltacabtagene autoleucel demonstrated unprecedented clinical activity in a heavily pretreated patient population, including with an ORR of 96.9% and rapid onset of response in less than 1 month.

### 6.5. Example 5. Efficacy Outcomes and Characteristics of Patients with Multiple Myeloma (MM) Who Achieved Sustained Minimal Residual Disease Negativity after Treatment with Ciltacabtagene Autoleucel (Cilta-Cel)

Cilta-cel is a BCMA-targeting CAR T-cell therapy that was recently approved by the US FDA for the treatment of adult patients (pts) with relapsed/refractory multiple myeloma (RRMM) after ≥4 prior lines of therapy (LOT) including a proteasome inhibitor (PI), immunomodulatory drug (IMiD), and anti-CD38 monoclonal antibody. In the phase 1b/2 CARTITUDE-1 study, cilta-cel demonstrated deep and durable responses in heavily pretreated pts with RRMM. The ORR was 97.9%, and at a median follow-up of 28 months (mo) the DOR was not reached. Of the 97 pts, 61 were evaluable for minimal residual disease (MRD) negativity (clonoSEQ v2.0, Adaptive Biotechnologies), with 56 (91.8%) of these 61 pts achieving MRD negativity (10⁻⁵) at any point. MRD negativity was sustained for <6 months in 22 pts, 6-12 months in 10 pts, and ≥12 months in 24 pts. This analysis sought to characterize the baseline and disease characteristics of pts with sustained MRD negativity (pts who continued to remain MRD negative 6 months and 12 months).

Eligible pts had MM and received 3 prior therapies or were refractory to a PI, and IMiD, and had received a PI, IMiD, and an anti-CD38 antibody. Pts received a single cilta-cel infusion (target dose 0.75×10⁶ CAR+ viable T cells/kg) 5-7 days post lymphodepletion. MRD negativity was a secondary objective of the phase 2 part of CARTITUDE-1 and was assessed on bone marrow samples at baseline; day 28; and 6, 12, 18, and 24 month using next-generation sequencing, regardless of disease status. An additional sample was collected and assessed at the time of suspected complete response and every 12 months until progressive disease (PD) for pts who remained on the study. Evaluable samples passed calibration and quality control and included sufficient cells for evaluation at 10⁻⁵ testing threshold. Characteristics were analyzed in pts who had MRD negativity<6 months, or sustained 6 months and 12 months. Pts who did not achieve MRD negativity at any time point were considered to be MRD positive. Landmark analyses were conducted at 6 and 12 months to address immortal time bias.

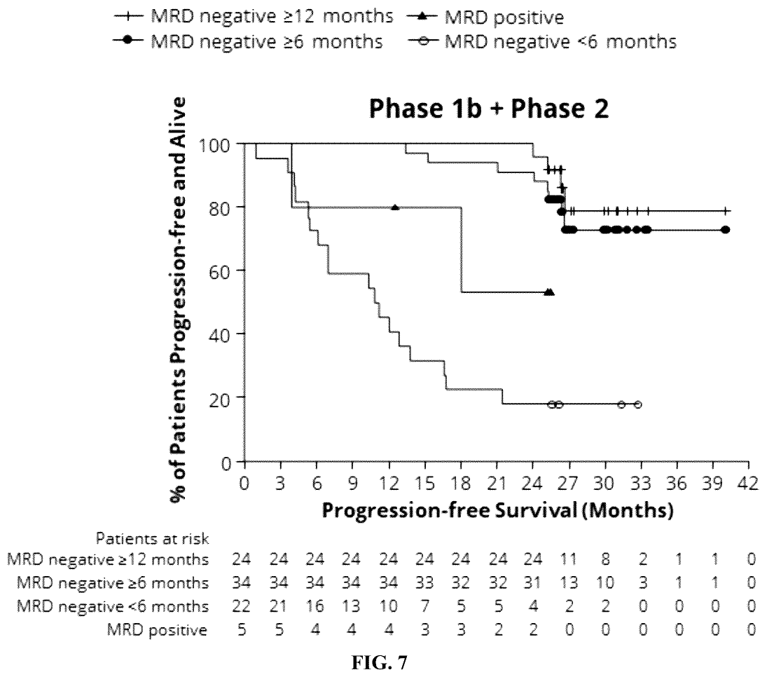

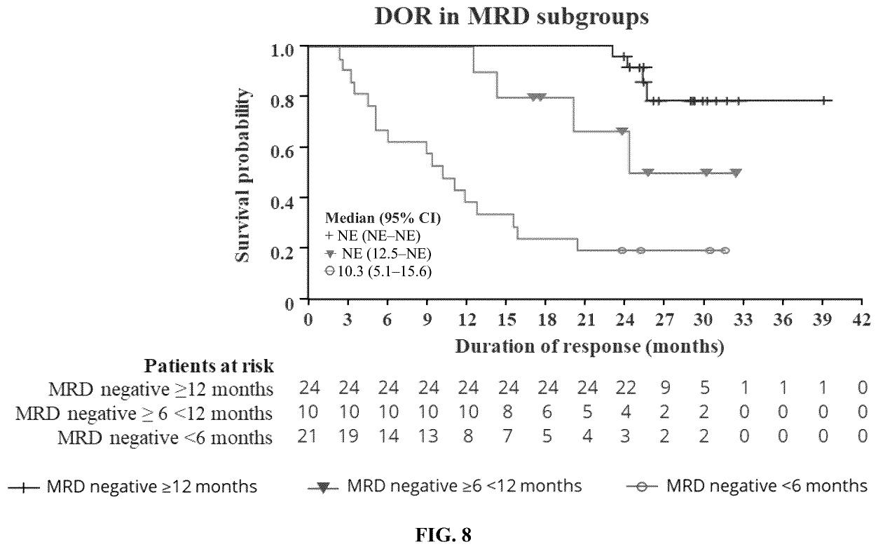

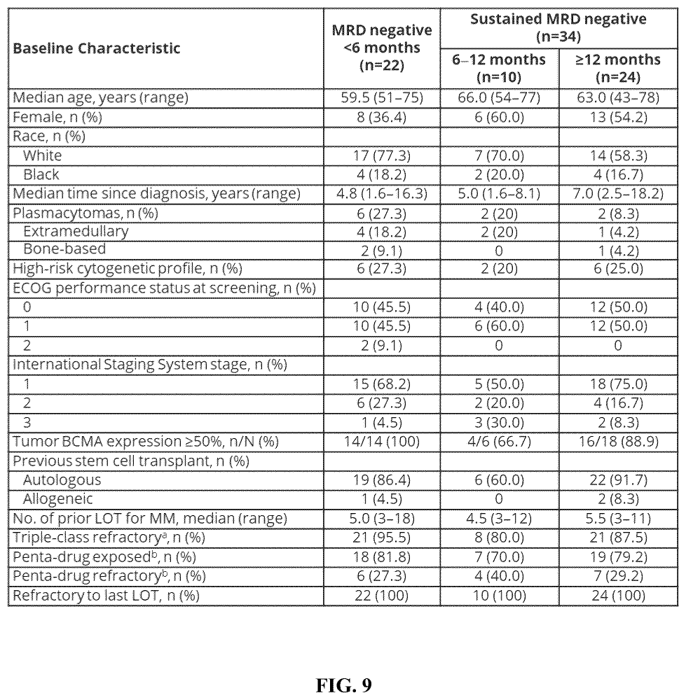

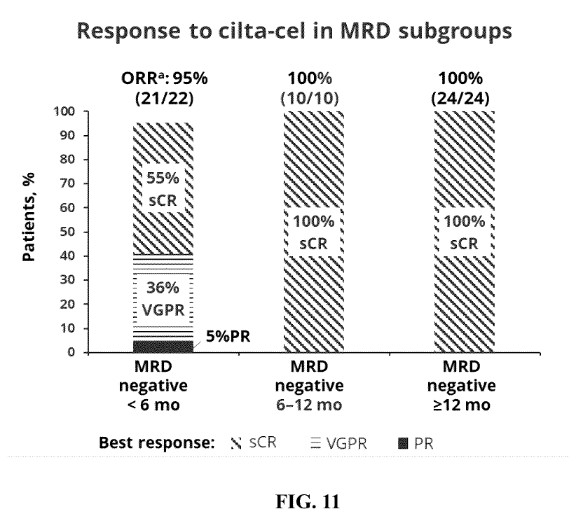

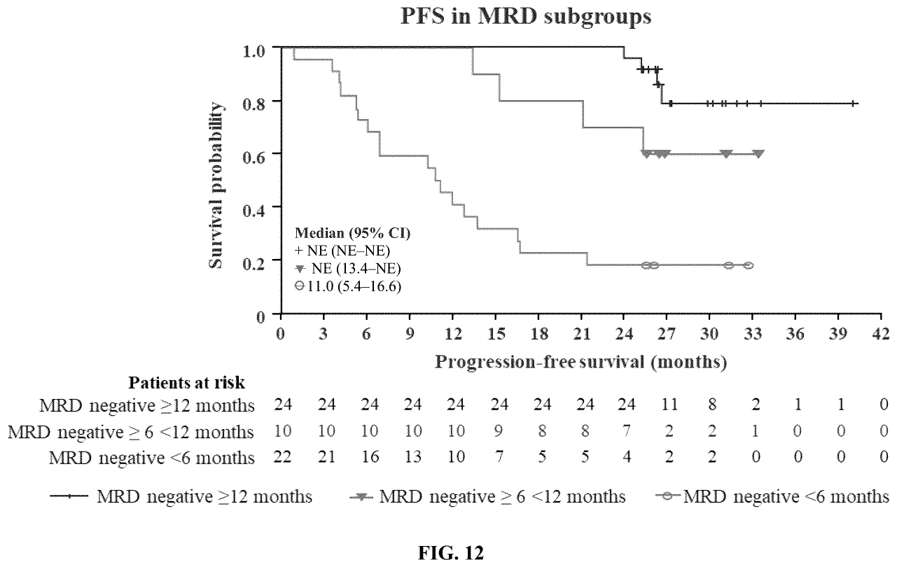

Of the 56 pts who achieved MRD negativity in CARTITUDE-1, 50 had at least 6 months follow-up without progression after initial MRD-negativity and 44 had at least 12 months follow-up without progression after initial MRD-negativity. MRD negativity was sustained for ≥6 months in 68% (34 of 50 with at least 6 months follow-up without progression after initial MRD-negativity) and ≥12 months in 55% (24 of 44 with at least 12 months follow-up without progression after initial MRD-negativity; all 6 pts evaluable for sustained MRD at ≥6 months but not≥12 months had PD). Pts with sustained MRD negativity (≥6 months, and ≥12 months) had longer progression free survival (PFS; FIGS. 7 and 12) compared with pts who did not (MRD negative<6 months; consistent with results from landmark analyses). The duration of response in MRD subgroups are shown in FIG. 8. Responders who had sustained MRD negativity had longer DOR compared with those who did not. Key pt and disease characteristics in these different groups (FIG. 9), and potential associations with sustained MRD negativity were analyzed descriptively. Baseline extramedullary plasmacytomas were less common in pts with MRD sustained for ≥6 months (8.8%, n=3/34) and ≥12 months (4.2%, n=1/24) compared with pts who had MRD negativity<6 months (18.2%, n=4/22). All Pts with sustained MRD negativity for ≥6 months achieved stringent complete response (sCR) (FIG. 11). Pts with sustained MRD negativity also had trends toward longer median time since diagnosis (5.9 years and 7.0 years for pts with MRD negativity sustained≥6 and ≥12 months, respectively versus 4.8 years for those with MRD negativity<6 months). Overall, pts who achieved sustained MRD negativity for ≥, 6 months had deeper responses than those with MRD negativity sustained for <6 months. Other baseline characteristics, including presence of high-risk cytogenetics, ECOG performance status, ISS stage, number of priorLOT, penta-drug refractoriness, and refractoriness to last LOT did not differ across MRD subgroups, and were similar to the overall CARTITUDE-1 population.

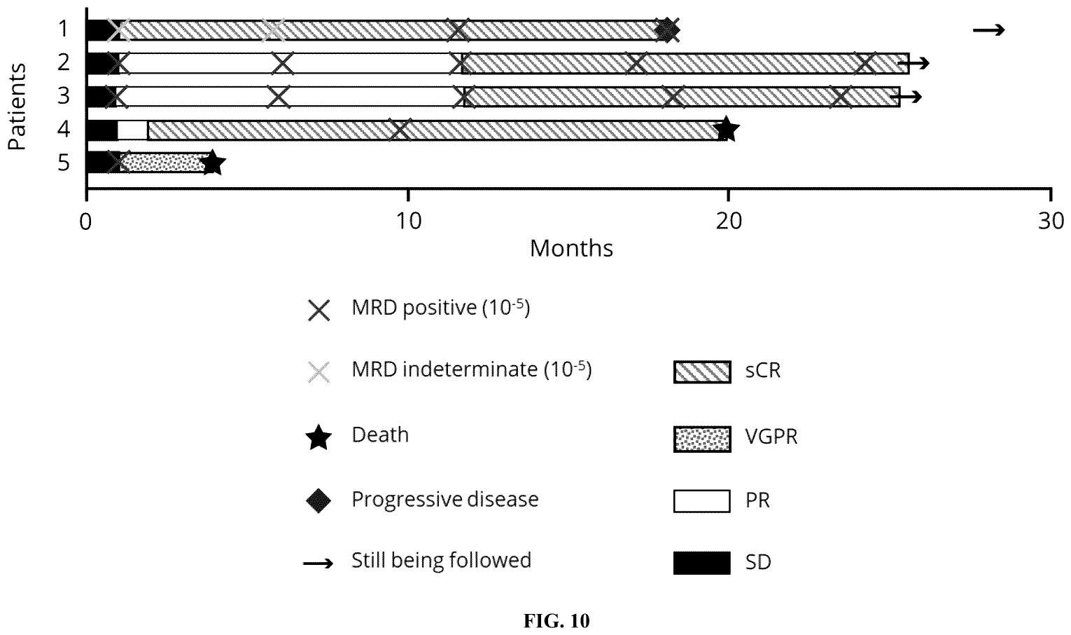

Based on our descriptive analysis, pts receiving cilta-cel achieved MRD negativity irrespective of their high-risk cytogenetics and ISS status, number of prior LOT and penta-drug refractoriness. While MRD negativity alone (<6 months group) did not appear to provide PFS benefit when compared to MRD positive pts, the small number of pts in the MRD positive group (FIG. 10) prevents comparisons with those who achieved MRD negativity. Generally, patients who did not achieve MRD negativity at any point (MRD positive) responded to cilta-cel. Presence of extramedullary plasmacytoma at baseline, and time since diagnosis might be factors that impact achievement of sustained MRD negativity. These data suggest that while cilta-cel is effective for a broad range of pts, specific pt and disease characteristics may be associated with sustained MRD negativity and better long-term outcomes.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

### Tables

TABLE 1
Summary of Subject Treatment Overview; All Enrolled Analysis Set

| | | | Phase 1b + |
|---|---|---|---|
| | Phase 1b | Phase 2 | Phase 2 |
| Analysis set: all enrolled | 35 | 78 | 113 |
| Subject who underwent apheresis | 35 (100.0%) | 78 (100.0%) | 113 (100.0%) |
| Subjects who received conditioning | 30 (85.7%) | 71 (91.0%) | 101 (89.4%) |
| regimen | | | |
| Subjects who received cilta-cel infusion | 29 (82.9%) | 68 (87.2%) | 97 (85.8%) |
| Subjects received conditioning regimen | 1 (2.9%) | 3 (3.8%) | 4 (3.5%) |
| but did not receive cilta-cel infusion | | | |
| Reasons | | | |
| Adverse event | 1 (2.9%) | 0 | 1 (0.9%) |
| Subject refused further study treatment | 0 | 2 (2.6%) | 2 (1.8%) |
| Death | 0 | 1 (1.3%) | 1 (0.9%) |

TABLE 2
Summary of Study Duration of Follow-up; All Treated Analysis Set

| | | | Phase 1b + |
|---|---|---|---|
| | Phase 1b | Phase 2 | Phase 2 |
| Analysis set: all treated | 29 | 68 | 97 |
| Duration of follow-up (months) | | | |
| N | 29 | 68 | 97 |
| Mean (SD) | 16.67 (3.815) | 10.79 (2.597) | 12.55 (4.033) |
| Median | 16.94 | 11.27 | 12.42 |
| Range | (3.3+; 24.9) | (1.5+; 14.8) | (1.5+; 24.9) |

+ Denotes subjects who died.

TABLE 3
Summary of Prior Therapies for Multiple Myeloma; All Treated Analysis Set

| | | | Phase 1b + |
|---|---|---|---|
| | Phase 1b | Phase 2 | Phase 2 |
| Analysis set: all treated | 29 | 68 | 97 |
| Number of lines of prior therapies | | | |
| for multiple myeloma | | | |
| N | 29 | 68 | 97 |
| Category, n (%) | | | |
| 3 | 7 (24.1%) | 10 (14.7%) | 17 (17.5%) |
| 4 | 3 (10.3%) | 13 (19.1%) | 16 (16.5%) |
| 5 | 6 (20.7%) | 9 (13.2%) | 15 (15.5%) |
| >5 | 13 (44.8%) | 36 (52.9%) | 49 (50.5%) |
| Mean (SD) | 6.1 (3.37) | 6.4 (3.19) | 6.3 (3.23) |
| Median | 5.0 | 6.0 | 6.0 |
| Range | (3; 18) | (3; 18) | (3; 18) |
| Prior transplantation | 26 (89.7%) | 61 (89.7%) | 87 (89.7%) |
| Autologous | 26 (89.7%) | 61 (89.7%) | 87 (89.7%) |
| 1 | 19 (65.5%) | 51 (75.0%) | 70 (72.2%) |
| 2 | 7 (24.1%) | 10 (14.7%) | 17 (17.5%) |
| Allogenic | 0 | 8 (11.8%) | 8 (8.2%) |
| Prior radiotherapy | 7 (24.1%) | 40 (58.8%) | 47 (48.5%) |
| Prior cancer-related | 2 (6.9%) | 22 (32.4%) | 24 (24.7%) |
| surgery/procedure | | | |
| Prior PI | 29 (100.0%) | 68 (100.0%) | 97 (100.0%) |
| Bortezomib | 25 (86.2%) | 67 (98.5%) | 92 (94.8%) |
| Carfilzomib | 26 (89.7%) | 57 (83.8%) | 83 (85.6%) |
| Ixazomib | 9 (31.0%) | 20 (29.4%) | 29 (29.9%) |
| Prior IMiD | 29 (100.0%) | 68 (100.0%) | 97 (100.0%) |
| Lenalidomide | 29 (100.0%) | 67 (98.5%) | 96 (99.0%) |
| Pomalidomide | 26 (89.7%) | 63 (92.6%) | 89 (91.8%) |
| Thalidomide | 6 (20.7%) | 15 (22.1%) | 21 (21.6%) |
| Prior PI and Prior IMiD | 29 (100.0%) | 68 (100.0%) | 97 (100.0%) |
| Prior corticosteroids | 29 (100.0%) | 68 (100.0%) | 97 (100.0%) |
| Dexamethasone | 29 (100.0%) | 68 (100.0%) | 97 (100.0%) |
| Prednisone | 3 (10.3%) | 6 (8.8%) | 9 (9.3%) |
| Prior alkylating agents | 28 (96.6%) | 66 (97.1%) | 94 (96.9%) |
| Prior anthracyclines | 9 (31.0%) | 18 (26.5%) | 27 (27.8%) |
| Prior anti-CD38 antibodies | 29 (100.0%) | 68 (100.0%) | 97 (100.0%) |
| Daratumumab | 27 (93.1%) | 67 (98.5%) | 94 (96.9%) |
| Isatuximab | 2 (6.9%) | 6 (8.8%) | 8 (8.2%) |
| TAK-079 | 1 (3.4%) | 0 | 1 (1.0%) |
| Prior Elotuzumab | 4 (13.8%) | 19 (27.9%) | 23 (23.7%) |
| Prior Panobinostat | 5 (17.2%) | 6 (8.8%) | 11 (11.3%) |
| Prior PI + IMiD + ALKY | 28 (96.6%) | 66 (97.1%) | 94 (96.9%) |
| Prior PI + IMiD + anti-CD38 antibodies | 29 (100.0%) | 68 (100.0%) | 97 (100.0%) |
| Prior PI + IMiD + anti-CD38 | 28 (96.6%) | 66 (97.1%) | 94 (96.9%) |
| antibodies + ALKY | | | |
| Prior penta-exposed (at least 2 PIs + | 22 (75.9%) | 59 (86.8%) | 81 (83.5%) |
| at least 2 IMiDs + 1 anti-CD38 | | | |
| antibodies) | | | |

TABLE 4
Summary of Refractory Status to Prior Multiple
Myeloma Therapy; All Treated Analysis Set

| | | | Phase 1b + |
|---|---|---|---|
| | Phase 1b | Phase 2 | Phase 2 |
| Analysis set: all treated | 29 | 68 | 97 |
| Refractory at any point to prior therapy | 29 (100.0%) | 68 (100.0%) | 97 (100.0%) |
| Refractory Status | | | |
| PI + IMiD + anti-CD38 antibody | 25 (86.2%) | 60 (88.2%) | 85 (87.6%) |
| Any PI | 25 (86.2%) | 62 (91.2%) | 87 (89.7%) |
| Any IMiD | 28 (96.6%) | 67 (98.5%) | 95 (97.9%) |
| Any anti-CD38 antibody | 29 (100.0%) | 67 (98.5%) | 96 (99.0%) |
| At least 2 PIs + at least 2 IMiDs + | 9 (31.0%) | 32 (47.1%) | 41 (42.3%) |
| 1 anti-CD38 antibody | | | |
| Refractory to last line of prior therapy | 28 (96.6%) | 68 (100.0%) | 96 (99.0%) |
| Refractory to | | | |
| Bortezomib | 15 (51.7%) | 51 (75.0%) | 66 (68.0%) |
| Carfilzomib | 21 (72.4%) | 42 (61.8%) | 63 (64.9%) |
| Ixazomib | 7 (24.1%) | 20 (29.4%) | 27 (27.8%) |
| Lenalidomide | 22 (75.9%) | 57 (83.8%) | 79 (81.4%) |
| Pomalidomide | 22 (75.9%) | 59 (86.8%) | 81 (83.5%) |
| Thalidomide | 1 (3.4%) | 7 (10.3%) | 8 (8.2%) |
| Daratumumab | 27 (93.1%) | 67 (98.5%) | 94 (96.9%) |
| Isatuximab | 2 (6.9%) | 5 (7.4%) | 7 (7.2%) |
| TAK-079 | 1 (3.4%) | 0 | 1 (1.0%) |
| Elotuzumab | 1 (3.4%) | 18 (26.5%) | 19 (19.6%) |
| Panobinostat | 3 (10.3%) | 5 (7.4%) | 8 (8.2%) |

Two additional subjects were refractory to other anti-CD38 antibodies

TABLE 5
Pre-infusion Medications

| Medication | Dose | Administration |
|---|---|---|
| Antihistamine | diphenhydramine (50 | Oral - administer 1 hour (±15 minutes) prior to cilta- |
| | mg) or equivalent | cel infusion |
| | | Or |
| | | IV- start infusion 30 minutes (±15 minutes) prior to |
| | | cilta-cel infusion |
| Antipyretic | acetaminophen (650 | Oral or IV - administer 30 minutes (±15 minutes) |
| | mg to 1,000 mg) or | prior to cilta-cel infusion |
| | equivalent | |

TABLE 6
Criteria for Response to Multiple Myeloma Treatment

| Response | Response Criteria |
|---|---|
| Stringent | CR as defined below, plus |
| complete | Normal FLC ratio, and |
| response | Absence of clonal plasma cells (PCs) by immunohistochemistry or 2- to |
| (sCR) | 4-color flow cytometry |
| Complete | Negative immunofixation of serum and urine, and |
| response | Disappearance of any soft tissue plasmacytomas, and |
| (CR)^{a} | <5% PCs in bone marrow |
| | No evidence of initial monoclonal protein isotype(s) on immunofixation |
| | of the serum and urine.^{b} |
| Very good | Serum and urine M-component detectable by immunofixation but not on |
| partial | electrophoresis, or |
| response | ≥90% reduction in serum M-component plus urine M-component <100 |
| (VGPR)^{a} | mg/24 hours |
| Partial | ≥50% reduction of serum M-protein and reduction in 24-hour urinary M- |
| response | protein by ≥90% or to <200 mg/24 hours |
| (PR) | If serum and urine M-protein were not measurable, a decrease ≥50% in |
| | the difference between involved and uninvolved FLC levels was |
| | required in place of the M-protein criteria |
| | If serum and urine M-protein were not measurable, and serum FLC assay |
| | was also not measurable, ≥50% reduction in bone marrow PCs was |
| | required in place of M-protein, provided baseline percentage had been |
| | ≥30% |
| | In addition to the above criteria, if present at baseline, ≥50% reduction |
| | in the size of soft tissue plasmacytomas was also required. |
| Minimal | ≥25% but ≤49% reduction of serum M-protein and reduction in 24-hour |
| response | urine M-protein by 50% to 89% |
| (MR) | In addition to the above criteria, if present at baseline, ≥50% reduction |
| | in the size of soft tissue plasmacytomas was also required. |
| Stable | Not meeting criteria for sCR, CR, VGPR, PR, MR, or progressive |
| disease | disease |
| Progressive | Any one or more of the following criteria: |
| disease^{c} | Increase of 25% from lowest response value in any of the following: |
| | Serum M-component (absolute increase must be ≥0.5 g/dL), and/or |
| | Urine M-component (absolute increase must be ≥200 mg/24 hours), |
| | and/or |
| | Only in subjects without measurable serum and urine M-protein |
| | levels: the difference between involved and uninvolved FLC levels |
| | (absolute increase must be >10 mg/dL) |
| | Only in subjects without measurable serum and urine M-protein |
| | levels and without measurable disease by FLC levels, bone marrow |
| | PC percentage (absolute increase must be ≥10%). |
| | Appearance of a new lesion(s), ≥50% increase from nadir in sum of the |
| | products of the maximal perpendicular diameters of measured lesions |
| | of >1 lesion, or ≥50% increase in the longest diameter of a previous |
| | lesion >1 cm in short axis |
| | Definite development of new bone lesions or definite increase in the size |
| | of existing bone lesions |
| | ≥50% increase in circulating plasma cells (minimum of 200 cells per μL) |
| | if this was the only measure of disease |

^{a}Clarifications to the criteria for coding CR and VGPR in subjects in whom the only measurable disease is by serum FLC levels: CR in such subjects indicates a normal FLC ratio of 0.26 to 1.65 in addition to CR criteria listed above. VGPR in such subjects requires a ≥90% decrease in the difference between involved and uninvolved FLC levels. For patients achieving very good partial response by other criteria, a soft tissue plasmacytoma must decrease by more than 90% in the sum of the maximal perpendicular diameter (SPD) compared with baseline.
^{b}In some cases it is possible that the original M protein light-chain isotype is still detected on immunofixation but the accompanying heavy-chain component has disappeared; this would not be considered as a CR even though the heavy-chain component is not detectable, since it is possible that the clone evolved to one that secreted only light chains. Thus, if a patient has IgA lambda myeloma, then to qualify as CR there should be no IgA detectable on serum or urine immunofixation; if free lambda is detected without IgA, then it must be accompanied by a different heavy chain isotype (IgG, IgM, etc.).
^{c}Clarifications to the criteria for coding progressive disease: bone marrow criteria for progressive disease are to be used only in subjects without measurable disease by M-protein and by FLC levels; “25% increase” refers to M-protein, and FLC, and does not refer to bone lesions, or soft tissue plasmacytomas and the “lowest response value” does not need to be a confirmed value.
Notes:
All response categories (CR, sCR, VGPR, PR, MR, and progressive disease) require 2 consecutive assessments made at any time before the institution of any new therapy; CR, sCR, VGPR, PR, MR, and stable disease categories also require no known evidence of progressive or new bone lesions if radiographic studies were performed. VGPR and CR categories require serum and urine studies regardless of whether disease at baseline was measurable on serum, urine, both, or neither.
Radiographic studies are not required to satisfy these response requirements. Bone marrow assessments need not be confirmed. For progressive disease, serum M-component increases of ≥1 g/dL are sufficient to define relapse if lowest M-component is ≥5 g/dL.

TABLE 7
Cytokine Release Syndrome ASTCT Consensus Grading System

| Grade | Toxicity |
|---|---|
| Grade 1 | Fever^{a} (Temperature ≥38°) |
| Grade 2 | Fever^{a} (Temperature ≥38°) with either: |
| | Hypotension not requiring vasopressors |
| | And/or^{c} hypoxia requiring low-flow nasal cannula^{b} or blow-by. |
| Grade 3 | Fever^{a} (Temperature ≥38°) with either: |
| | Hypotension requiring a vasopressor with or without |
| | vasopressin, |
| | And/or^{c} hypoxia requiring high-flow nasal cannula^{b}, facemask, |
| | nonrebreather mask, or Venturi mask. |
| Grade 4 | Fever^{a} (Temperature ≥38°) with either: |
| | hypotension requiring multiple vasopressors (excluding |
| | vasopressin), |
| | And/or^{c} hypoxia requiring positive pressure (eg, CPAP, BiPAP, |
| | intubation and mechanical ventilation). |
| Grade 5 | Death |

^{a}Fever not attributable to any other cause. In patients who have CRS then receive antipyretics or anticytokine therapy such as tocilizumab or steroids, fever is no longer required to grade subsequent CRS severity. In this case, CRS grading is driven by hypotension and/or hypoxia.
^{b}Low-flow nasal cannula is defined as oxygen delivered at ≤6 L/minute or blow-by oxygen delivery. High-flow nasal cannula is defined as oxygen delivered at >6 L/minute.
^{c}CRS grade is determined by the more severe event: hypotension or hypoxia not attributable to any other cause.
Note:
Organ toxicities associated with CRS may be graded according to CTCAE v5.0 but they do not influence CRS grading.

TABLE 8
Immune Effector Cell-associated Neurotoxicity Syndrome
(ICANS) ASTCT Consensus Grading System^{a, b}

| Neurotoxicity | | | | |
|---|---|---|---|---|
| Domain | Grade 1 | Grade 2 | Grade 3 | Grade 4 |
| ICE Score | 7-9 | 3-6 | 0-2 | 0 (patient is |
| | | | | unarousable and |
| | | | | unable to |
| | | | | perform ICE). |
| Depressed | Awakens | Awakens | Awakens only to | Patient is |
| Level of | spontaneously. | to voice. | tactile stimulus. | unarousable or |
| Consciousness | | | | requires |
| | | | | vigorous or |
| | | | | repetitive tactile |
| | | | | stimuli |
| | | | | to arouse. |
| | | | | Stupor or |
| | | | | coma. |
| Seizure | N/A | N/A | Any clinical seizure, | Life-threatening |
| | | | focal or generalized, | prolonged |
| | | | that | seizure (>5 |
| | | | resolves rapidly; or | min); or |
| | | | Non-convulsive | Repetitive |
| | | | seizures on EEG that | clinical or |
| | | | resolve with | electrical |
| | | | intervention. | seizures |
| | | | | without return |
| | | | | to |
| | | | | baseline in |
| | | | | between. |
| Motor | N/A | N/A | N/A | Deep focal |
| Findings | | | | motor |
| | | | | weakness such |
| | | | | as |
| | | | | hemiparesis or |
| | | | | paraparesis. |
| Raised | N/A | N/A | Focal/local edema | Diffuse cerebral |
| Intracranial | | | on neuroimaging. | edema on |
| Pressure / | | | | neuroimaging; |
| Cerebral | | | | or |
| Edema | | | | Decerebrate or |
| | | | | decorticate |
| | | | | posturing; or |
| | | | | Cranial nerve |
| | | | | VI palsy; or |
| | | | | Papilledema; or |
| | | | | Cushing's triad. |

^{a}Toxicity grading according to Lee et al 2019
^{b}ICANS grade is determined by the most severe event (ICE score, level of consciousness, seizure, motor findings, raised ICP/cerebral edema) not attributable to any other cause.
Note:
all other neurological adverse events (not associated with ICANS) should continue to be graded with CTCAE Version 5.0 during both phases of the study

TABLE 9
Overall Best Response Based on International Myeloma Working Group (IMWG)
Consensus Criteria, as Assessed by Independent Review Committee (IRC);
All Treated Analysis Set at Median Follow-Up Time of 12.4 Months

| | Phase 1b | | Phase 2 | | Phase 1b + Phase 2 | |
|---|---|---|---|---|---|---|
| | | 95% exact | | 95% exact | | 95% exact |
| | n (%) | CI for % | n (%) | CI for % | n (%) | CI for % |
| Analysis set: all treated | 29 | | 68 | | 97 | |
| Best response | | | | | | |
| Stringent complete | 25 | (68.3%, | 40 | (46.2%, | 65 | (56.7%, |
| response (sCR) | (86.2%) | 96.1%) | (58.8%) | 70.6%) | (67.0%) | 76.2%) |
| Complete response | 0 | (NE, NE) | 0 | (NE, NE) | 0 | (NE, NE) |
| (CR) | | | | | | |
| MRD-negative | 14 | (29.4%, | 19 | (17.7%, | 33 | (24.7%, |
| CR/sCR ^{a} | (48.3%) | 67.5%) | (27.9%) | 40.1%) | (34.0%) | 44.3%) |
| Very good partial | 3 | (2.2%, | 22 | (21.5%, | 25 | (17.4%, |
| response (VGPR) | (10.3%) | 27.4%) | (32.4%) | 44.8%) | (25.8%) | 35.7%) |
| Partial response | 1 | (0.1%, | 3 | (0.9%, | 4 | (1.1%, |
| (PR) | (3.4%) | 17.8%) | (4.4%) | 12.4%) | (4.1%) | 10.2%) |
| Minimal response | 0 | (NE, NE) | 0 | (NE, NE) | 0 | (NE, NE) |
| (MR) | | | | | | |
| Stable disease | 0 | (NE, NE) | 0 | (NE, NE) | 0 | (NE, NE) |
| (SD) | | | | | | |
| Progressive disease | 0 | (NE, NE) | 1 | (0.0%, | 1 | (0.0%, |
| (PD) | | | (1.5%) | 7.9%) | (1.0%) | 5.6%) |
| Not evaluable (NE) | 0 | (NE, NE) | 2 | (0.4%, | 2 | (0.3%, |
| | | | (2.9%) | 10.2%) | (2.1%) | 7.3%) |
| Overall response | 29 | (88.1%, | 65 | (87.6%, | 94 | (91.2%, |
| (sCR + CR + | (100.0%) | 100.0%) | (95.6%) | 99.1%) | (96.9%) | 99.4%) |
| VGPR + PR) | | | | | | |
| P-value (one-sided, | | | | | <0.0001 | |
| exact binomial test | | | | | | |
| for null hypothesis | | | | | | |
| of overall response | | | | | | |
| rate ≤30%) | | | | | | |
| Clinical benefit | 29 | (88.1%, | 65 | (87.6%, | 94 | (91.2%, |
| (Overall | (100.0%) | 100.0%) | (95.6%) | 99.1%) | (96.9%) | 99.4%) |
| response + MR) | | | | | | |
| VGPR or better | 28 | (82.2%, | 62 | (81.8%, | 90 | (85.7%, |
| (sCR + | (96.6%) | 99.9%) | (91.2%) | 96.7%) | (92.8%) | 97.0%) |
| CR + VGPR) | | | | | | |
| CR or better | 25 | (68.3%, | 40 | (46.2%, | 65 | (56.7%, |
| (sCR + CR) | (86.2%) | 96.1%) | (58.8%) | 70.6%) | (67.0%) | 76.2%) |

Keys: CI = confidence interval.
^{a} MRD-negative CR/sCR. Only MRD assessments (10⁻⁵ testing threshold) within 3 months of achieving CR/sCR until death/progression/subsequent therapy (exclusive) were considered.

TABLE 10
Duration of Response Based on Independent Review Committee
(IRC) Assessment; Responders in All Treated Analysis
Set at Median Follow-Up Time of 12.4 Months

| | | | Phase 1b + |
|---|---|---|---|
| | Phase 1b | Phase 2 | Phase 2 |
| Analysis set: responders in all | 29 | 65 | 94 |
| treated | | | |
| Duration of response | | | |
| Number of events (%) | 9 (31.0%) | 15 (23.1%) | 24 (25.5%) |
| Number of censored (%) | 20 (69.0%) | 50 (76.9%) | 70 (74.5%) |
| Kaplan-Meier estimate (months) | | | |
| 25% quantile (95% CI) | 12.0 (6.0, NE) | 10.3 (4.5, NE) | 11.1 (6.0, NE) |
| Median (95% CI) | NE (15.9, NE) | NE (NE, NE) | NE (15.9, NE) |
| 75% quantile (95% CI) | NE (NE, NE) | NE (NE, NE) | NE (NE, NE) |
| 6-month event-free rate % | 93.1 (75.1, 98.2) | 80.7 (68.5, 88.5) | 84.6 (75.4, 90.6) |
| (95% CI) | | | |
| 9-month event-free rate % | 86.2 (67.3, 94.6) | 77.4 (64.8, 85.9) | 80.2 (70.4, 87.0) |
| (95% CI) | | | |
| 12-month event-free rate % | 72.1 (51.8, 85.0) | 71.9 (54.8, 83.4) | 68.2 (54.4, 78.6) |
| (95% CI) | | | |

Key: CI = confidence interval, NE = Not estimable.

TABLE 11
Progression-Free Survival Based on Independent Review Committee (IRC) Assessment;
All Treated Analysis Set at Median Follow-Up Time of 12.4 Months

| | | | Phase 1b + |
|---|---|---|---|
| | Phase 1b | Phase 2 | Phase 2 |
| Analysis set: all treated | 29 | 68 | 97 |
| Progression-free survival | | | |
| Number of events (%) | 9 (31.0%) | 16 (23.5%) | 25 (25.8%) |
| Number of censored (%) | 20 (69.0%) | 52 (76.5%) | 72 (74.2%) |
| Kaplan-Meier estimate (months) | | | |
| 25% quantile (95% CI) | 13.73 (6.93, NE) | 11.17 (5.42, NE) | 12.02 (6.97, NE) |
| Median (95% CI) | NE (16.79, NE) | NE (NE, NE) | NE (16.79, NE) |
| 75% quantile (95% CI) | NE (NE, NE) | NE (NE, NE) | NE (NE, NE) |
| 6-month progression-free survival rate % | 93.1 (75.1, 98.2) | 85.3 (74.4, 91.8) | 87.6 (79.2, 92.8) |
| (95% CI) | | | |
| 9-month progression-free survival rate % | 86.2 (67.3, 94.6) | 77.8 (65.9, 86.0) | 80.3 (70.9, 87.0) |
| (95% CI) | | | |
| 12-month progression-free survival rate % | 82.8 (63.4, 92.4) | 72.6 (56.5, 83.6) | 76.6 (66.0, 84.3) |
| (95% CI) | | | |
| 18-month progression-free survival rate % | 57.7 (25.9, 79.9) | NE (NE, NE) | 54.2 (26.4, 75.4) |
| (95% CI) | | | |

Key: CI = confidence interval.

TABLE 12
Overall Survival; All Treated Analysis Set at Median Follow-Up Time of 12.4 Months

| | | | Phase 1b + |
|---|---|---|---|
| | Phase 1b | Phase 2 | Phase 2 |
| Analysis set: all treated | 29 | 68 | 97 |
| Overall survival | | | |
| Number of events (%) | 5 (17.2%) | 9 (13.2%) | 14 (14.4%) |
| Number of censored (%) | 24 (82.8%) | 59 (86.8%) | 83 (85.6%) |
| Kaplan-Meier estimate (months) | | | |
| 25% quantile (95% CI) | 19.12 (13.73, NE) | NE (NE, NE) | 19.12 (19.12, NE) |
| Median (95% CI) | 22.80 (19.12, NE) | NE (NE, NE) | 22.80 (19.12, NE) |
| 75% quantile (95% CI) | NE (22.80, NE) | NE (NE, NE) | NE (22.80, NE) |
| 6-month overall survival rate % | 96.6 (77.9, 99.5) | 92.6 (83.2, 96.9) | 93.8 (86.7, 97.2) |
| (95% CI) | | | |
| 9-month overall survival rate % | 93.1 (75.1, 98.2) | 89.7 (79.5, 94.9) | 90.7 (82.8, 95.0) |
| (95% CI) | | | |
| 12-month overall survival rate % | 93.1 (75.1, 98.2) | 86.5 (75.7, 92.7) | 88.5 (80.2, 93.5) |
| (95% CI) | | | |
| 18-month overall survival rate % | 89.7 (71.3, 96.5) | NE (NE, NE) | 85.8 (75.4, 92.1) |
| (95% CI) | | | |

Key: CI = confidence interval.

TABLE 13
Summary of Overall Minimal Residual Disease (MRD) Negativity Rate
at 10⁻⁵ in Bone Marrow Based on Next-Generation Sequencing (NGS);
All Treated Analysis Set at Median Follow-Up Time of 12.4 Months

| | | | Phase 1b + |
|---|---|---|---|
| | Phase 1b | Phase 2 | Phase 2 |
| Analysis set: all treated | 29 | 68 | 97 |
| MRD negativity rate (10) | 18 (62.1%) | 35 (51.5%) | 53 (54.6%) |
| 95% exact CI of MRD negative | (42.3%, 79.3%) | (39.0%, 63.8%) | (44.2%, 64.8%) |
| rate | | | |

Key: CI = confidence interval.

TABLE 14
Summary of Overall Minimal Residual Disease (MRD) Negativity Rate at 10⁻⁵
in Bone Marrow Based on Next-Generation Sequencing; Subjects with Evaluable Sample
at 10⁻⁵ in All Treated Analysis Set at Median Follow-Up Time of 12.4 Months

| | | | Phase 1b + |
|---|---|---|---|
| | Phase 1b | Phase 2 | Phase 2 |
| Analysis set: subjects with evaluable | 18 | 39 | 57 |
| sample at 10in all treated | | | |
| MRD negativity rate (10) | 18 (100.0%) | 35 (89.7%) | 53 (93.0%) |
| 95% exact CI of MRD negative rate | (81.5%, 100.0%) | (75.8%, 97.1%) | (83.0%, 98.1%) |

Key: CI = confidence interval.

TABLE 15
Summary of Treatment-emergent Cytokine Release
Syndrome (CRS) Events; All Treated Analysis
Set at Median Follow-Up Time of 12.4 Months

| | | | Phase 1b + |
|---|---|---|---|
| | Phase 1b | Phase 2 | Phase 2 |
| Analysis set: all treated | 29 | 68 | 97 |
| Number of subjects with | 27 (93.1%) | 65 (95.6%) | 92 (94.8%) |
| CRS | | | |
| Maximum toxicity grade | | | |
| Grade 1 | 14 (48.3%) | 35 (51.5%) | 49 (50.5%) |
| Grade 2 | 10 (34.5%) | 28 (41.2%) | 38 (39.2%) |
| Grade 3 | 1 (3.4%) | 2 (2.9%) | 3 (3.1%) |
| Grade 4 | 1 (3.4%) | 0 | 1 (1.0%) |
| Grade 5 | 1 (3.4%) | 0 | 1 (1.0%) |
| Time from initial infusion | | | |
| of CAR-T cells to first | | | |
| onset of CRS (days) | | | |
| N | 27 | 65 | 92 |
| Mean (SD) | 7.0 (2.01) | 6.4 (2.28) | 6.6 (2.21) |
| Median | 7.0 | 7.0 | 7.0 |
| Range | (2; 12) | (1; 10) | (1; 12) |
| Duration of CRS (days) | | | |
| N | 27 | 65 | 92 |
| Mean (SD) | 7.0 (18.04) | 5.2 (2.68) | 5.7 (9.94) |
| Median | 3.0 | 4.0 | 4.0 |
| Range | (2; 97) | (1; 14) | (1; 97) |
| Interquartile range | (2.0; 4.0) | (3.0; 6.0) | (3.0; 6.0) |
| Number of subjects with | 26 (89.7%) | 62 (91.2%) | 88 (90.7%) |
| supportive measures to | | | |
| treat CRS | | | |
| Anti-IL6 receptor | 23 (79.3%) | 44 (64.7%) | 67 (69.1%) |
| Tocilizumab | | | |
| IL-1 receptor antagonist | 6 (20.7%) | 12 (17.6%) | 18 (18.6%) |
| Anakinra | | | |
| Corticosteroids | 6 (20.7%) | 15 (22.1%) | 21 (21.6%) |
| Vasopressor used | 2 (6.9%) | 2 (2.9%) | 4 (4.1%) |
| Oxygen used | 1 (3.4%) | 5 (7.4%) | 6 (6.2%) |
| Blow-by | 0 | 0 | 0 |
| Nasal cannula low flow | 1 (3.4%) | 5 (7.4%) | 6 (6.2%) |
| (≤6 L/min) | | | |
| Nasal cannula high flow | 0 | 1 (1.5%) | 1 (1.0%) |
| (>6 L/min) | | | |
| Face mask | 0 | 0 | 0 |
| Non-Rebreather mask | 0 | 0 | 0 |
| Venturi mask | 0 | 0 | 0 |
| Other | 0 | 0 | 0 |
| Positive pressure | 1 (3.4%) | 0 | 1 (1.0%) |
| Bilevel Positive Airway | 1 (3.4%) | 0 | 1 (1.0%) |
| Pressure | | | |
| Intubation/ Mechanical | 1 (3.4%) | 0 | 1 (1.0%) |
| Ventilation | | | |
| Other | 24 (82.8%) | 57 (83.8%) | 81 (83.5%) |
| Outcome of CRS | | | |
| N | 27 | 65 | 92 |
| Recovered or resolved | 26 (96.3%) | 65 (100.0%) | 91 (98.9%) |
| Not recovered or not | 0 | 0 | 0 |
| resolved | | | |
| Recovered or resolved with | 0 | 0 | 0 |
| sequelae | | | |
| Recovering or resolving | 0 | 0 | 0 |
| Fatal | 1 (3.7%) | 0 | 1 (1.1%) |
| Unknown | 0 | 0 | 0 |

TABLE 16
Summary of Immune Effector Cell-Associated Neurotoxicity (ICANS)
With Onset After Ciltacabtagene Autoleucel Infusion; All Treated
Analysis Set at Median Follow-Up Time of 12.4 Months

| | | | Phase 1b + |
|---|---|---|---|
| | Phase 1b | Phase 2 | Phase 2 |
| Analysis set: all treated | 29 | 68 | 97 |
| Number of subjects with ICANS | 3 (10.3%) | 13 (19.1%) | 16 (16.5%) |
| Maximum toxicity grade | | | |
| Grade 1 | 2 (6.9%) | 8 (11.8%) | 10 (10.3%) |
| Grade 2 | 0 | 4 (5.9%) | 4 (4.1%) |
| Grade 3 | 1 (3.4%) | 0 | 1 (1.0%) |
| Grade 4 | 0 | 1 (1.5%) | 1 (1.0%) |
| Grade 5 | 0 | 0 | 0 |
| Time from initial infusion of | | | |
| cilta-cel to first onset of | | | |
| ICANS | | | |
| N | 3 | 13 | 16 |
| Mean (SD) | 6.3 (2.89) | 7.5 (2.22) | 7.3 (2.29) |
| Median | 8.0 | 8.0 | 8.0 |
| Range | (3; 8) | (4; 12) | (3; 12) |
| Duration of ICANS (days) | | | |
| N | 3 | 13 | 16 |
| Mean (SD) | 3.7 (2.08) | 5.2 (3.09) | 4.9 (2.93) |
| Median | 3.0 | 4.0 | 4.0 |
| Range | (2; 6) | (1; 12) | (1; 12) |
| Number of subjects with | 3 (10.3%) | 13 (19.1%) | 16 (16.5%) |
| treatment of ICANS | | | |
| IL-1 receptor antagonist | 0 | 3 (4.4%) | 3 (3.1%) |
| anakinra | | | |
| Anti-IL6 receptor tocilizumab | 1 (3.4%) | 2 (2.9%) | 3 (3.1%) |
| Corticosteroid | 1 (3.4%) | 8 (11.8%) | 9 (9.3%) |
| Levetiracetam | 0 | 1 (1.5%) | 1 (1.0%) |
| Dexamethasone | 1 (3.4%) | 8 (11.8%) | 9 (9.3%) |
| Methylprednisolone sodium | 0 | 1 (1.5%) | 1 (1.0%) |
| succinate | | | |
| Pethidine | 0 | 1 (1.5%) | 1 (1.0%) |
| Outcome of ICANS | | | |
| N | 3 | 13 | 16 |
| Recovered or resolved | 3 (100.0%) | 13 (100.0%) | 16 (100.0%) |

TABLE 17
Summary of Cytopenias Following Treatment With Ciltacabtagene Autoleucel;
All Treated Analysis Set at Median Follow-Up Time of 12.4 Months

| | Phase 1b + Phase 2 | | |
|---|---|---|---|
| | (N = 97) | | |
| | | Initial Grade 3/4 (%) | Initial Grade 3/4 (%) |
| | Grade 3/4 (%) After | Recovered to <=Grade | Recovered to <=Grade |
| | Day 1 Dosing | 2 by Day 30 | 2 by Day 60 |
| Thrombocytopenia | 60 (61.9%) | 23 (23.7%) | 41 (42.3%) |
| Neutropenia | 95 (97.9%) | 67 (69.1%) | 85 (87.6%) |
| Lymphopenia | 96 (99.0%) | 84 (86.6%) | 88 (90.7%) |

SEQUENCES
Ciltacabtagene autoleucel CAR CD8α
signal peptide, CD8α SP amino
acid sequence
SEQ ID NO: 1
MALPVTALLLPLALLLHAARP
Ciltacabtagene autoleucel CAR BCMA
binding domain, VHH1 amino
acid sequence
SEQ ID NO: 2
QVKLEESGGGLVQAGRSLRLSCAASEHTFSSHVMGWFRQAPGKER
ESVAVIGWRDISTSYADSVKGRFTISRDNAKKTLYLQMNSLKPE
DTAVYYCAARRIDAADFDSWGQGTQVTVSS
Ciltacabtagene autoleucel CAR BCMA
binding domain, G4S linker
amino acid sequence
SEQ ID NO: 3
GGGGS
Ciltacabtagene autoleucel CAR BCMA
binding domain, VHH2 amino
acid sequence
SEQ ID NO: 4
EVQLVESGGGLVQAGGSLRLSCAASGRTFTMGWFRQAPGKEREFV
AAISLSPTLAYYAESVKGRFTISRDNAKNTVVLQMNSLKPEDTAL
YYCAADRKSVMSIRPDYWGQGTQVTVSS
Ciltacabtagene autoleucel CAR CD8α
hinge amino acid sequence
SEQ ID NO: 5
TTTPAPRPPTPAPTIASQPLSLRPEACRPAAGGAVHTRGLDFACD
Ciltacabtagene autoleucel CAR CD8α
transmembrane amino acid
sequence
SEQ ID NO: 6
IYIWAPLAGTCGVLLLSLVITLYC
Ciltacabtagene autoleucel CAR CD137
Cytoplasmic amino acid sequence
SEQ ID NO: 7
KRGRKKLLYIFKQPFMRPVQTTQEEDGCSCRFPEEEEGGCEL
Ciltacabtagene autoleucel CAR CD3ζ
Cytoplasmic amino acid sequence
SEQ ID NO: 8
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR
Ciltacabtagene autoleucel CAR CD8α
signal peptide CD8a SP nucleic
acid sequence
SEQ ID NO: 9
ATGGCTCTGCCCGTCACCGCTCTGCTGCTGCCTCTGGCTCTGCTG
CTGCACGCTGCTCGCCCT
Ciltacabtagene autoleucel CAR BCMA
binding domain, VHH1 nucleic
acid sequence
SEQ ID NO: 10
CAGGTCAAACTGGAAGAATCTGGCGGAGGCCTGGTGCAGGCAGGA
CGGAGCCTGCGCCTGAGCTGCGCAGCATCCGAGCACACCTTCAGC
TCCCACGTGATGGGCTGGTTTCGGCAGGCCCCAGGCAAGGAGAGA
GAGAGCGTGGCCGTGATCGGCTGGAGGGACATCTCCACATCTTAC
GCCGATTCCGTGAAGGGCCGGTTCACCATCAGCCGGGACAACGCC
AAGAAGACACTGTATCTGCAGATGAACAGCCTGAAGCCCGAGGAC
ACCGCCGTGTACTATTGCGCAGCAAGGAGAATCGACGCAGCAGAC
TTTGATTCCTGGGGCCAGGGCACCCAGGTGACAGTGTCTAGC
Ciltacabtagene autoleucel CAR BCMA
binding domain, G4S linker
nucleic acid sequence
SEQ ID NO: 11
GGAGGAGGAGGATCT
Ciltacabtagene autoleucel CAR BCMA
binding domain, VHH2 nucleic
acid sequence
SEQ ID NO: 12
GAGGTGCAGCTGGTGGAGAGCGGAGGCGGCCTGGTGCAGGCCGGA
GGCTCTCTGAGGCTGAGCTGTGCAGCATCCGGAAGAACCTTCACA
ATGGGCTGGTTTAGGCAGGCACCAGGAAAGGAGAGGGAGTTCGTG
GCAGCAATCAGCCTGTCCCCTACCCTGGCCTACTATGCCGAGAGC
GTGAAGGGCAGGTTTACCATCTCCCGCGATAACGCCAAGAATACA
GTGGTGCTGCAGATGAACTCCCTGAAACCTGAGGACACAGCCCTG
TACTATTGTGCCGCCGATCGGAAGAGCGTGATGAGCATTAGACCA
GACTATTGGGGGCAGGGAACACAGGTGACCGTGAGCAGC
Ciltacabtagene autoleucel CAR CD8α
hinge nucleic acid sequence
SEQ ID NO: 13
ACCACGACGCCAGCGCCGCGACCACCAACACCGGCGCCCACCATC
GCGTCGCAGCCCCTGTCCCTGCGCCCAGAGGCGTGCCGGCCAGCG
GCGGGGGGCGCAGTGCACACGAGGGGGCTGGACTTCGCCTGTGAT
Ciltacabtagene autoleucel CAR CD8α
transmembrane nucleic acid
sequence
SEQ ID NO: 14
ATCTACATCTGGGCGCCCTTGGCCGGGACTTGTGGGGTCCTTCTC
CTGTCACTGGTTATCACCCTTTACTGC
Ciltacabtagene autoleucel CAR CD137
Cytoplasmic nucleic acid
sequence
SEQ ID NO: 15
AAACGGGGCAGAAAGAAACTCCTGTATATATTCAAACAACCATTT
ATGAGACCAGTACAAACTACTCAAGAGGAAGATGGCTGTAGCTGC
CGATTTCCAGAAGAAGAAGAAGGAGGATGTGAACTG
Ciltacabtagene autoleucel CAR CD3ζ
Cytoplasmic nucleic acid
sequence
SEQ ID NO: 16
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGAGAAGGAAGAACCCTCAGGAAGGCCTGTACAAT
GAACTGCAGAAAGATAAGATGGCGGAGGCCTACAGTGAGATTGGG
ATGAAAGGCGAGCGCCGGAGGGGCAAGGGGCACGATGGCCTTTAC
CAGGGTCTCAGTACAGCCACCAAGGACACCTACGACGCCCTTCAC
ATGCAGGCCCTGCCCCCTCGCTAA
Ciltacabtagene autoleucel CAR amino
acid sequence
SEQ ID NO: 17
MALPVTALLLPLALLLHAARPQVKLEESGGGLVQAGRSLRLSCAA
SEHTFSSHVMGWFRQAPGKERESVAVIGWRDISTSYADSVKGRFT
ISRDNAKKTLYLQMNSLKPEDTAVYYCAARRIDAADFDSWGQGTQ
VTVSSGGGGSEVQLVESGGGLVQAGGSLRLSCAASGRTFTMGWFR
QAPGKEREFVAAISLSPTLAYYAESVKGRFTISRDNAKNTVVLQM
NSLKPEDTALYYCAADRKSVMSIRPDYWGQGTQVTVSSTSTTTPA
PRPPTPAPTIASQPLSLRPEACRPAAGGAVHTRGLDFACDIYIWA
PLAGTCGVLLLSLVITLYCKRGRKKLLYIFKQPFMRPVQTTQEED
GCSCRFPEEEEGGCELRVKFSRSADAPAYQQGQNQLYNELNLGRR
EEYDVLDKRRGRDPEMGGKPRRKNPQEGLYNELQKDKMAEAYSEI
GMKGERRRGKGHDGLYQGLSTATKDTYDALHMQALPPR
Ciltacabtagene autoleucel CAR BCMA
binding domain, VHH1 CDR1
SEQ ID NO: 18
SHVMG
Ciltacabtagene autoleucel CAR BCMA
binding domain, VHH1 CDR2
SEQ ID NO: 19
VIGWRDISTSYADSVKG
Ciltacabtagene autoleucel CAR BCMA
binding domain, VHH1 CDR3
SEQ ID NO: 20
ARRIDAADFDS
Ciltacabtagene autoleucel CAR BCMA
binding domain, VHH2 CDR1
SEQ ID NO: 21
TFTMG
Ciltacabtagene autoleucel CAR BCMA
binding domain, VHH2 CDR2
SEQ ID NO: 22
AISLSPTLAYYAESVKG
Ciltacabtagene autoleucel CAR BCMA
binding domain, VHH2 CDR3
SEQ ID NO: 23
ADRKSVMSIRPDY

1. A method for assessing responsiveness of a subject to a treatment comprising T cells expressing a bivalent BCMA-targeting chimeric antigen receptor (CAR), comprising:
(a) administering to the subject the T cells;
(b) measuring time length the subject maintains minimal residual disease (MRD) negative status; and
(c) assessing the responsiveness of the subject to the treatment based on that
(i) the MRD negative status is maintained for shorter than 6 months;
(ii) the MRD negative status is maintained for at least 6 months and shorter than 12 months; or
(iii) the MRD negative status is maintained for at least 12 months, wherein the bivalent BCMA-targeting CAR comprises an extracellular antigen binding domain comprising a first VHH domain and a second VHH domain, a transmembrane domain, and an intracellular signaling domain, wherein the first VHH domain comprising a CDR1, a CDR2, and a CDR3 as set forth in the VHH domain comprising the amino acid sequence of SEQ ID NO: 2, and the second VHH domain comprising a CDR1, a CDR2, and a CDR3 as set forth in the VHH domain comprising the amino acid sequence of SEQ ID NO: 4.

2. The method of claim 1, wherein the first VHH domain comprises a CDR1 comprising the amino acid sequence of SEQ ID NO: 18, a CDR2 comprising the amino acid sequence of SEQ ID NO: 19, a CDR3 comprising the amino acid sequence of SEQ ID NO: 20; and the second VHH domain comprises a CDR1 comprising the amino acid sequence of SEQ ID NO: 21, a CDR2 comprising the amino acid sequence of SEQ ID NO: 22, and a CDR3 comprising the amino acid sequence of SEQ ID NO: 23.

3. The method of claim 1, wherein the first VHH domain comprises the amino acid sequence of SEQ ID NO: 2 and the second VHH domain comprises the amino acid sequence of SEQ ID NO: 4.

4. The method of claim 1, wherein the first VHH domain is at the N-terminus of the second VHH domain, or the first VHH domain is at the C-terminus of the second VHH domain, wherein optionally the first VHH domain is linked to the second VHH domain via a linker comprising the amino acid sequence of SEQ ID NO: 3.

5. (canceled)

6. The method of claim 1, wherein the transmembrane domain is derived from a molecule selected from the group consisting of CD8a, CD4, CD28, CD137, CD80, CD86, CD152 and PD1, wherein optionally the transmembrane domain is derived from CD8a and comprises the amino acid sequence of SEQ ID NO: 6.

7. (canceled)

8. The method of claim 1, wherein:
(1) the intracellular signaling domain comprises a primary intracellular signaling domain of an immune effector cell, wherein optionally the primary intracellular signaling domain is derived from CD3ζ comprising the amino acid sequence of SEQ ID NO: 8: or
(2) the intracellular signaling domain comprises a co-stimulatory signaling domain, wherein optionally the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and any combination thereof, wherein further optionally the co-stimulatory signaling domain comprises a cytoplasmic domain of CD137 comprising the amino acid sequence of SEQ ID NO: 7.

9-12. (canceled)

13. The method of claim 1, wherein the CAR further comprises:
(1) a hinge domain located between the C-terminus of the extracellular antigen binding domain and the N-terminus of the transmembrane domain, wherein optionally the hinge domain is derived from CD8a comprising the amino acid sequence of SEQ ID NO: 5; or
(2) a signal peptide located at the N-terminus of the polypeptide, wherein optionally the signal peptide is derived from CD8a comprising the amino acid sequence of SEQ ID NO: 1.

14-16. (canceled)

17. The method of claim 1, wherein the CAR comprises the amino acid sequence of SEQ ID NO: 17.

18. The method of claim 1, wherein the subject has a disease or disorder, wherein optionally the disease or disorder is cancer, wherein further optionally the cancer is multiple myeloma, and wherein further optionally the cancer is refractory or relapsed multiple myeloma.

19-21. (canceled)

22. The method of claim 1, wherein the method comprises obtaining bone marrow aspirate or biopsy from the subject for assessing MRD status, wherein optionally the MRD status is monitored using next generation sequencing (NGS) of bone marrow aspirate DNA, wherein further optionally the NGS is performed via clonoSEQ.

23-24. (canceled)

25. The method of claim 22, wherein baseline bone marrow aspirates are used to define the myeloma clones, and post-treatment samples are used to evaluate MRD negativity, wherein optionally evaluable samples are those that passed one or more of, or all of, calibration, quality control, and sufficiency of cells evaluable at a particular sensitivity level, wherein further optionally the sensitivity level is about 10⁻⁶, 10⁻⁵, 10⁻⁴, or 10⁻³.

26-27. (canceled)

28. The method of claim 1, wherein the time length the subject maintains MRD negative status is measured from the time when MRD is first achieved in the subject.

29. The method of claim 1, wherein the method comprises assessing the likelihood of the subject to have complete response (CR), partial response (PR), stringent CR (sCR), or very good PR (VGPR).

30. The method of claim 29, comprising determining that the subject is likely to have CR, PR, sCR, or VGPR if the MRD negative status is maintained for shorter than 6 months in the subject, or determining that the subject is likely to have CR or sCR if the MRD negative status is maintained for longer than 6 months in the subject.

31. (canceled)

32. The method of claim 1, wherein the method comprises assessing the likelihood of the subject to have progression-free survival.

33. The method of claim 32, comprising determining that the subject is likely to have progression-free survival for at least 12 months post the treatment if the MRD negative status is maintained for longer than 6 months in the subject.

34. The method of claim 32, comprising determining that the subject is likely to have progression-free survival for at least 24 months post the treatment if the MRD negative status is maintained for longer than 12 months in the subject.

35. The method of claim 1, wherein the method comprises assessing duration of response in the subject.

36. The method of claim 35, comprising determining that the subject is likely to have a duration of response of more than 12 months if the MRD negative status is maintained for longer than 6 months in the subject.

37. The method of claim 36, comprising determining that the subject is likely to have a duration of response of more than 24 months if the MRD negative status is maintained for longer than 12 months in the subject.
